SEQUENCE LISTING (2 sequences)

SEQ ID NO: 1 - protein, 10 aa, synthetic construct
  REGION 1..10: note = RSK2 peptide
KRRRLSSLRA

SEQ ID NO: 2 - protein, 12 aa, synthetic construct
  REGION 1..12: note = Abl peptide
EAIYAAPFAKKK

(19) United States
(12) Patent Application Publication
(10) Pub. No.: US 2024/0002392 A1
(43) Pub. Date: Jan. 4, 2024

(54) Azetidinyl Pyrimidines and Uses Thereof

(71) Applicant: Aerie Pharmaceuticals, Inc., Bedminster, NJ (US)

(72) Inventors: David Archer Ellis, Bedminster, NJ (US); Heeren M. Gordhan, Durham, NC (US); Cynthia L. Lichorowic, Durham, NC (US); Jill M. Sturdivant, Chapel Hill, NC (US); Mitchell A. deLong, Chapel Hill, NC (US)

(21) Appl. No.: 18/344,405

(22) Filed: Jun. 29, 2023

Related U.S. Application Data
(60) Provisional application No. 63/356,670, filed on Jun. 29, 2022.

(51) Int. Cl. C07D 487/04; C07D 417/14; C07D 401/14; C07D 403/14

(52) U.S. Cl. CPC C07D 487/04; C07D 417/14; C07D 401/14; C07D 403/14

(57) ABSTRACT

Provided herein are compounds useful as inhibitors of Janus kinase (JAK) proteins and in treating JAK-related diseases.

### RELATED APPLICATIONS

This application claims priority of U.S. Provisional Patent Application No. 63/356,670, filed Jun. 29, 2022, the entire content of which is incorporated herein by reference.

### SEQUENCE LISTING

This application contains a sequence listing having the filename 1960110_00620_SL.xml, which is 3,315 bytes in size, and was created on May 30, 2023. The entire content of this sequence listing is incorporated herein by reference.

### BACKGROUND

Janus kinase (JAK) proteins are a family of cytoplasmic protein tyrosine kinases. The four JAKs, JAK1, JAK2, JAK3, and TYK2, and seven signal transducer and activator of transcription (STAT) transcription factors mediate intracellular signal transduction downstream of cytokine receptors, which are implicated in the pathology of autoimmune, allergic, and inflammatory diseases, among others. The JAKs usually associate with cytokine receptors in pairs as either homodimers or heterodimers. Specific cytokines are associated with specific JAK pairings. Each of the four members of the JAK family is implicated in the signaling pathways of at least one of the cytokines associated with inflammation. Binding of a cytokine to a JAK-dependent cytokine receptor induces receptor dimerization which results in phosphorylation of tyrosine residues on the JAK kinase, effecting JAK activation. Phosphorylated JAKs, in turn, bind and phosphorylate various STAT proteins which dimerize, translocate to the cell nucleus, and directly modulate gene transcription, leading, among other effects, to the downstream effects associated with inflammatory or autoimmune disease. Additionally, there is recent evidence that the kinases ROCK1 and ROCK2 may interact with the STAT enzymes, resulting in downregulation of IL-21 and IL-17 secretion. Inhibition of ROCK kinases has been noted to have an anti-inflammatory effect as well as the well-known effects of JAK inhibition. In view of the role that kinases play in many disease states, there is an urgent and continuing need for small molecule ligands which inhibit or modulate the activity of kinases.

### SUMMARY

Described herein are compounds, having a formula:

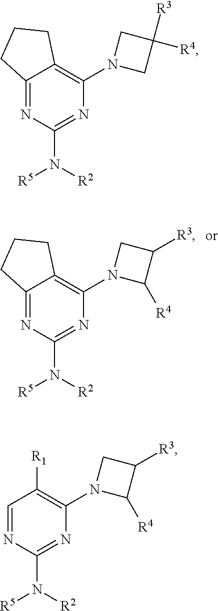

or a pharmaceutically acceptable salt thereof.

Also described herein are uses of the compounds, including inhibition of kinases and treatment of related diseases.

### BRIEF DESCRIPTION OF THE DRAWINGS

FIG. 1 shows a general scheme for preparing the compounds described herein, where R¹, R², R³, R⁴, and R⁵ are as defined herein.

### DETAILED DESCRIPTION

Described herein are azetidinyl pyrimidine compounds that affect the function of kinases and other proteins, which are useful in treating diseases.

### Definitions

Certain terms, whether used alone or as part of a phrase or another term, are defined below.

The articles “a” and “an” refer to one or to more than one of the grammatical object of the article.

Numerical values relating to measurements are subject to measurement errors that place limits on their accuracy. For this reason, all numerical values provided herein, unless otherwise indicated, are to be understood as being modified by the term “about.” Accordingly, the last decimal place of a numerical value provided herein indicates its degree of accuracy. Where no other error margins are given, the maximum margin is ascertained by applying the rounding-off convention to the last decimal place or last significant digit when a decimal is not present in the given numerical value.

The term “alkyl” or “alkylene” refers to branched or straight chain saturated hydrocarbon.

The term “alkynyl” refers to an unsaturated hydrocarbon that includes at least one carbon-carbon triple bond.

The term “amelioration” means a lessening of severity of at least one indicator of a condition or disease, such as a delay or slowing in the progression of one or more indicators of a condition or disease. The severity of indicators may be determined by subjective or objective measures which are known to those skilled in the art.

The term “aryl” refers to a carbocyclic aromatic system comprising one, two, three, or more rings.

The term “Cn-m” refers to a moiety comprising n to m carbon atoms, wherein n and m are integers.

The terms “composition” and “pharmaceutical composition” refer to a mixture of at least one compound described herein with a pharmaceutically acceptable carrier. The pharmaceutical composition facilitates administration of the compound to a patient or subject. Multiple techniques of administering a compound exist including, but not limited to, intravenous, oral, aerosol, parenteral, ophthalmic, nasal, pulmonary, and topical administration.

The term “cycloalkyl” refers to a cyclic alkyl moiety comprising one, two, three, or more rings.

The terms “effective amount” and “therapeutically effective amount” refer to an amount of therapeutic compound, such as a compound described herein, administered to a subject, either as a single dose or as part of a series of doses, which is effective to produce a desired therapeutic effect. In general, the therapeutically effective amount can be estimated initially either in cell culture assays or in mammalian animal models, for example, in non-human primates, mice, rabbits, dogs, or pigs. The animal model may also be used to determine the appropriate concentration range and route of administration. Such information can then be used to determine useful doses and routes for administration in non-human subjects and human subjects.

The term “halo” or “halogen” refers to one or more atoms independently selected from F, Br, Cl, or I.

The term “haloalkyl” refers to an alkyl moiety substituted with one or more halogens.

The term “haloaryl” refers to an aryl moiety substituted with one or more halogens.

The term “halocycloalkyl” refers to a cycloalkyl moiety substituted with one or more halogens.

The term “heteroaryl” refers to an aryl moiety comprising at least one ring heteroatom selected from O, S, or N, wherein each ring may comprise, independently, one, two, three, or four ring heteroatoms independently selected from O, S, or N.

The term “heterocycloalkyl” refers to an cycloalkyl moiety comprising at least one ring heteroatom selected from O, S, or N, wherein each ring may comprise, independently, one, two, three, or four ring heteroatoms independently selected from O, S, or N.

The term “heterocyclyl” refers to a ring system comprising at least one heteroatom selected from O, S, or N, one or more rings, wherein each ring may comprise, independently, one, two, three, or four ring heteroatoms independently selected from O, S, or N.

The term “pharmaceutically acceptable carrier” means a pharmaceutically acceptable material, composition or carrier, such as a liquid filler, solid filler, stabilizer, dispersing agent, suspending agent, diluent, excipient, thickening agent, solvent, or encapsulating material, involved in carrying or transporting at least one compound described herein within or to the patient such that the compound may perform its intended function. A given carrier must be “acceptable” in the sense of being compatible with the other ingredients of a particular formulation, including the compounds described herein, and not injurious to the patient. Other ingredients that may be included in the pharmaceutical compositions described herein are known in the art and described, for example, in “Remington's Pharmaceutical Sciences” (Genaro (Ed.), Mack Publishing Co., 1985), the entire content of which is incorporated herein by reference.

The term “refractory disease” refers to a disease that continues to progress during treatment with a pharmaceutical ingredient other than the compounds provided herein, partially responds to the other treatment, or transiently responds to the other treatment. The term may be applied to each of the diseases referred to herein.

The terms “treatment” or “treating” refer to the application of one or more specific procedures used for the amelioration of a disease. A “prophylactic” treatment, refers to reducing the rate of progression of the disease or condition being treated, delaying the onset of that disease or condition, or reducing the severity of its onset.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the described subject matter and does not pose a limitation on the scope of the subject matter otherwise claimed. No language in the specification should be construed as indicating any non-claimed element essential to practicing the described subject matter.

Groupings of alternative elements or embodiments of this disclosure are not to be construed as limitations. Each group member may be referred to and claimed individually or in any combination with other members of the group or other elements found herein. Furthermore, a recited member of a group may be included in, or excluded from, another recited group for reasons of convenience or patentability. When any such inclusion or exclusion occurs, the specification is deemed to contain the group as modified thus fulfilling the written description of all Markush groups used in the appended claims.

References have been made to patents and printed publications throughout this specification, each of which are individually incorporated herein by reference in their entirety.

It is to be understood that the embodiments of this disclosure are illustrative. Accordingly, the present disclosure is not limited to that precisely as shown and described. Compounds

It has been found that the compounds described herein are useful as inhibitors of kinases, including JAK proteins.

Thus, provided herein are compounds, having a formula:

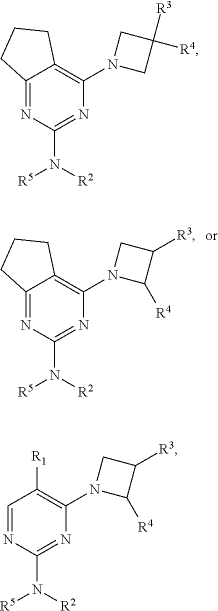

or a pharmaceutically acceptable salt thereof;
wherein
R⁵ is H or S(O)₂—(C₁₋₆ alkyl);
R¹ is H, CN, halogen, C₁₋₆ alkyl, O—(C₁₋₆ alkyl), or C₃₋₇ cycloalkyl;
R² is C₆₋₁₆ aryl or C₂₋₁₅ heterocyclyl, each of which may be substituted with 1 or 2 groups selected, independently, from OH, halogen, C₁₋₆ alkyl, (C₁₋₆ alkylene)-OH, O—(C₁₋₆ alkyl), (C₁₋₆ alkylene)-O—(C₁₋₆ alkyl), S(O)₂—(C₁₋₆ alkyl), C₃₋₇ cycloalkyl, (C₁₋₁₆ alkylene)-C(O)OH, (C₁₋₁₆ alkylene)-C(O)N(H)—(C₁₋₆ alkyl), (C₁₋₁₆ alkylene)-C(O)N(H)—OH, or (C₁₋₆ alkylene)-N(C₁₋₆ alkyl)-(C₁₋₆ alkyl);
R³ is CN, OH, NH₂, C₁₋₆ alkyl, C₁₋₆ alkynyl, C₃₋₇ cycloalkyl, (C₁₋₃ alkylene)-CN, (C₁₋₃ alkylene)-NH₂, (C₁₋₃ alkylene)-N(H)C(O)—(C₃₋₇ cycloalkyl), (C₁₋₃ alkylene)-N(H)C(O)—(C₂₋₈ heterocycloalkyl), O—(C₃₋₇ cycloalkyl), O—(C₁₋₃ alkylene)-CN, N(H)C(O)—(C₃₋₇ cycloalkyl), N(H)C(O)—(C₂₋₈ heterocycloalkyl), N(H)C(O)—(C₂₋₅ heteroaryl), N(H)C(O)—(C₁₋₃ alkylene)-(C₂₋₅ heteroaryl), N(H)C(O)—(C₆₋₁₀ aryl), N(H)C(O)—(C₁₋₃ alkylene)-(C₆₋₁₀ aryl), N(H)C(O)NH₂, N(H)(C₁₋₆ alkyl), N(H)(C₃₋₇ cycloalkyl), N(C₁₋₆ alkyl)(C₃₋₇ cycloalkyl), N(H)(C₁₋₃ alkylene)-(C₂₋₈ heterocycloalkyl), N(H)(C₁₋₃ alkylene)-(C₂₋₅ heteroaryl), N(H)(C₁₋₃ alkylene)-(C₆₋₁₀ aryl), or C₂₋₁₅ heterocyclyl, each of which may be substituted with 1, 2, 3, or 4 groups selected, independently, from halogen, O, OH, O—(C₁₋₆ alkyl), S(O)₂—(C₁₋₆ alkyl), S(O)₂—(C₁₋₆ haloalkyl), C(NH₂)(NH), C₁₋₆ alkyl, C₁₋₆ haloalkyl, (C₁₋₆ alkylene)-OH, (C₁₋₆ alkylene)-O—(C₁₋₆ alkyl), (C₁₋₆ alkylene)-O—(C₁₋₆ haloalkyl), (C₁₋₆ alkylene)-S(O)₂—(C₁₋₆ alkyl), (C₁₋₆ alkylene)-NH₂, (C₁₋₆ alkylene)-N(H)(C₁₋₆ alkyl), (C₁₋₆ alkylene)-N(C₁₋₆ alkyl)(C₁₋₆ alkyl), (C₁₋₆ alkylene)-(C₃₋₇ cycloalkyl), (C₁₋₆ alkylene)-(C₃₋₇ halocycloalkyl), (C₁₋₆ alkylene)-(C₂₋₅ heteroaryl), (C₁₋₆ alkylene)-(C₆₋₁₀ haloaryl), NH₂, N(H)(C₁₋₆ alkyl), N(C₁₋₆ alkyl)(C₁₋₆ alkyl), N(H)—(C₁₋₃ alkylene)-(C₃₋₇ cycloalkyl), N(C₁₋₆ alkyl)-(C₁₋₃ alkylene)-(C₃₋₇ cycloalkyl), N(H)C(O)O—(C₁₋₆ alkyl), N(H)S(O)₂—(C₁₋₆ alkyl), C(O)—(C₁₋₆ alkyl), C(O)—(C₁₋₆ alkylene)-CN, C(O)—(C₁₋₆ alkylene)-NH₂, C(O)—(C₁₋₆ alkylene)-N(H)(C₁₋₆ alkyl), C(O)—(C₁₋₆ alkylene)-N(C₁₋₆ alkyl)(C₁₋₆ alkyl), C(O)—(C₁₋₆ alkylene)-N(H)[S(O)₂—C₁₋₆ alkyl], C(O)—(C₁₋₆ alkylene)-N(C₁₋₆ alkyl)[S(O)₂—C₁₋₆ alkyl], or C(O)O—(C₁₋₆ alkyl); and
R⁴ is H, OH, C₁₋₆ alkyl, (C₁₋₆ alkylene)-OH, C₁₋₆ haloalkyl, C₃₋₇ cycloalkyl, C₃₋₇ halocycloalkyl, (C₁₋₃ alkylene)-(C₃₋₇ cycloalkyl), (C₁₋₆ alkylene)CN, (C₁₋₆ alkylene)-C(O)O—(C₁₋₆ alkyl), (C₁₋₆ alkylene)-OC(O)—(C₁₋₆ alkyl), or C₂₋₈ heterocycloalkyl;
or R³ and R⁴, together with the atoms to which they are attached, combine to form C₃₋₇ cycloalkyl, C₂₋₈ heterocycloalkyl, CC(H)—(C₀₋₆ alkylene)CN, C₃₋₇ cycloalkyl substituted with 1 or 2 groups selected, independently, from halogen or (C₁₋₆ alkylene)CN, or C₂₋₈ heterocycloalkyl substituted with 1 or 2 groups selected, independently, from halogen or (C₁₋₆ alkylene)CN.

In some embodiments, R⁵ is H.

In some embodiments, R² is phenyl, thiazolyl, isothiazolyl, pyrazolyl, imidazolyl, pyridinyl, pyrimidinyl, indolinyl, isoindolinyl, indolyl, phenylmorpholinyl, phenyloxetanyl, phenylpiperazinyl, dihydrobenzoborolyl, or benzoborolyl, each of which may be substituted with 1 or 2 groups selected, independently, from OH, F, Cl, Br, C₁₋₃ alkyl, (C₁₋₃ alkyl)-OH, C₁₋₆ haloalkyl, O—(C₁₋₃ alkyl), (C₁₋₃ alkylene)-O—(C₁₋₃ alkyl), S(O)₂—(C₁₋₃ alkyl), C₃₋₅ cycloalkyl, (C₆₋₁₆ alkylene)-C(O)OH, (C₆₋₁₆ alkylene)-C(O)N(H)—(C₁₋₆ alkyl), (C₆₋₁₆ alkylene)-C(O)N(H)—OH, or (C₁₋₃ alkylene)-N(C₁₋₃ alkyl)-(C₁₋₃ alkyl).

In some embodiments, R³ is CN, OH, NH₂, C₁₋₆ alkyl, C₁₋₆ alkynyl, C₃₋₇ cycloalkyl, (C₁₋₃ alkylene)-CN, or (C₁₋₃ alkylene)-NH₂,

In some embodiments, R³ is OH, NH₂, C₁₋₃ alkyl, C₁₋₃ alkynyl, C₃₋₇ cycloalkyl, or (C₁₋₃ alkylene)-NH₂, each of which may be substituted with 1, 2, or 3 groups selected, independently, from F, Cl, O, OH, O—(C₁₋₃ alkyl), C₁₋₃ alkyl, C₁₋₃ haloalkyl, (C₁₋₃ alkylene)-OH, (C₁₋₃ alkylene)-O—(C₁₋₃ alkyl), (C₁₋₃ alkylene)-NH₂, NH₂, N(H)(C₁₋₃ alkyl), or N(C₁₋₃ alkyl)(C₁₋₃ alkyl).

In some embodiments, R³ is (C₁₋₃ alkylene)-N(H)C(O)—(C₃₋₇ cycloalkyl), (C₁₋₃ alkylene)-N(H)C(O)—(C₂₋₈ heterocycloalkyl), O—(C₃₋₇ cycloalkyl), or O—(C₁₋₃ alkylene)-CN, each of which may be substituted with 1, 2, or 3 groups selected, independently, from F, Cl, O, OH, O—(C₁₋₃ alkyl), C₁₋₃ alkyl, C₁₋₃ haloalkyl, (C₁₋₃ alkylene)-OH, (C₁₋₃ alkylene)-O—(C₁₋₃ alkyl), (C₁₋₃ alkylene)-NH₂, NH₂, N(H)(C₁₋₃ alkyl), or N(C₁₋₃ alkyl)(C₁₋₃ alkyl).

In some embodiments, R³ is N(H)C(O)—(C₃₋₇ cycloalkyl), N(H)C(O)—(C₂₋₈ heterocycloalkyl), N(H)C(O)—(C₂₋₅ heteroaryl), N(H)C(O)—(C₁₋₃ alkylene)-(C₂₋₈ heteroaryl), N(H)C(O)—(C₆₋₁₀ aryl), N(H)C(O)—(C₁₋₃ alkylene)-(C₆₋₁₀ aryl), N(H)C(O)NH₂, N(H)(C₁₋₆ alkyl), N(H)(C₃₋₇ cycloalkyl), N(C₁₋₆ alkyl)(C₃₋₇ cycloalkyl), N(H)(C₁₋₃ alkylene)-(C₂₋₈ heterocycloalkyl), N(H)(C₁₋₃ alkylene)-(C₂₋₈ heteroaryl), or N(H)(C₁₋₃ alkylene)-(C₆₋₁₀ aryl), each of which may be substituted with 1, 2, or 3 groups selected, independently, from F, Cl, O, OH, O—(C₁₋₃ alkyl), C₁₋₃ alkyl, C₁₋₃ haloalkyl, (C₁₋₃ alkylene)-OH, (C₁₋₃ alkylene)-O—(C₁₋₃ alkyl), (C₁₋₃ alkylene)-NH₂, NH₂, N(H)(C₁₋₃ alkyl), or N(C₁₋₃ alkyl)(C₁₋₃ alkyl).

In some embodiments, R³ is C₂₋₁₅ heterocyclyl, which may be substituted with 1, 2, or 3 groups selected, independently, from F, Cl, O, OH, O—(C₁₋₃ alkyl), C₁₋₃ alkyl, C₁₋₃ haloalkyl, (C₁₋₃ alkylene)-OH, (C₁₋₃ alkylene)-O—(C₁₋₃ alkyl), (C₁₋₃ alkylene)-NH₂, NH₂, N(H)(C₁₋₃ alkyl), or N(C₁₋₃ alkyl)(C₁₋₃ alkyl).

In some embodiments, R³ and R⁴, together with the atoms to which they are attached, combine to form C₃₋₇ cycloalkyl, C₂₋₈ heterocycloalkyl, CC(H)—(C₀₋₃ alkylene)CN, C₃₋₇ cycloalkyl substituted with 1 or 2 groups selected, independently, from halogen or (C₁₋₃ alkylene)CN, or C₂₋₈ heterocycloalkyl substituted with 1 or 2 groups selected, independently, from halogen or (C₁₋₃ alkylene)CN.

In some embodiments, the compounds provided herein have a formula:

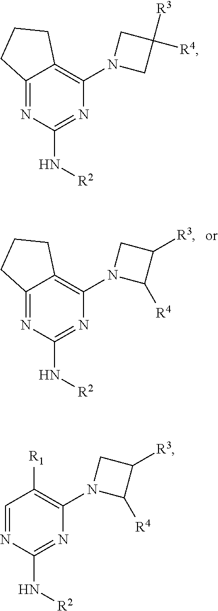

or a pharmaceutically acceptable salt thereof;
wherein
R¹ is H, F, Cl, Br, C₁₋₆ alkyl, or C₃₋₇ cycloalkyl;
R² is C₂₋₅ heteroaryl, or C₂₋₅ heteroaryl substituted with 1 or 2 groups selected, independently, from C₁₋₆ alkyl or (C₁₋₆ alkylene)OH;
R³ is C₂₋₈ heterocycloalkyl, or C₂₋₈ heterocycloalkyl substituted with 1 or 2 groups selected, independently, from C₁₋₆ alkyl, F, Cl, Br, or O(C₁₋₆ alkyl); and
R⁴ is (C₁₋₃ alkylene)CN.

In some embodiments, the compounds provided herein have a formula:

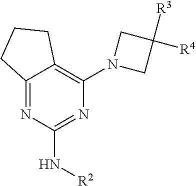

or a pharmaceutically acceptable salt thereof.

In some embodiments, the compounds provided herein have a formula:

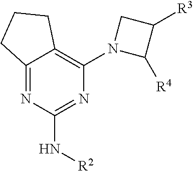

or a pharmaceutically acceptable salt thereof.

In some embodiments, the compounds provided herein have a formula:

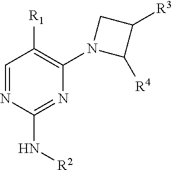

or a pharmaceutically acceptable salt thereof.

In some embodiments of the formulae provided herein, R¹ is F, Cl, Br, C₁₋₃ alkyl, or C₃₋₄ cycloalkyl. In some embodiments, R² is C₃₋₄ heteroaryl, or C₃₋₄ heteroaryl substituted with C₁₋₃ alkyl or (C₁₋₃ alkylene)OH. In some embodiments, R³ is C₄₋₈ heterocycloalkyl, or C₄₋₈ heterocycloalkyl substituted with 1 or 2 groups selected, independently, from C₁₋₃ alkyl, F, Cl, Br, or O(C₁₋₃ alkyl). In some embodiments, R⁴ is CH₂CN.

In some embodiments of the formulae provided herein, one or more of A)-F) applies:
A) R⁵ is H, S(O)₂CH₃, or S(O)₂CH₂CH₃;
B) R¹ is H, CH₃, CH₂CH₃, cyclopropyl, OCH₃, F, Cl, or CN;
C) R² is

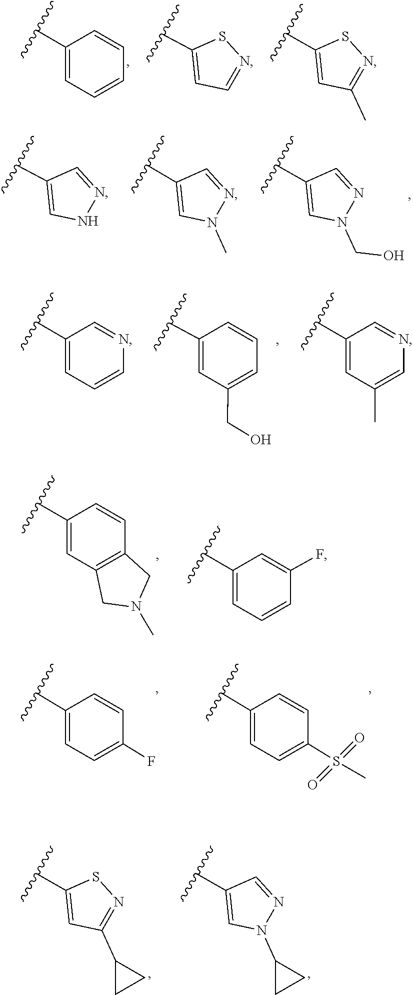

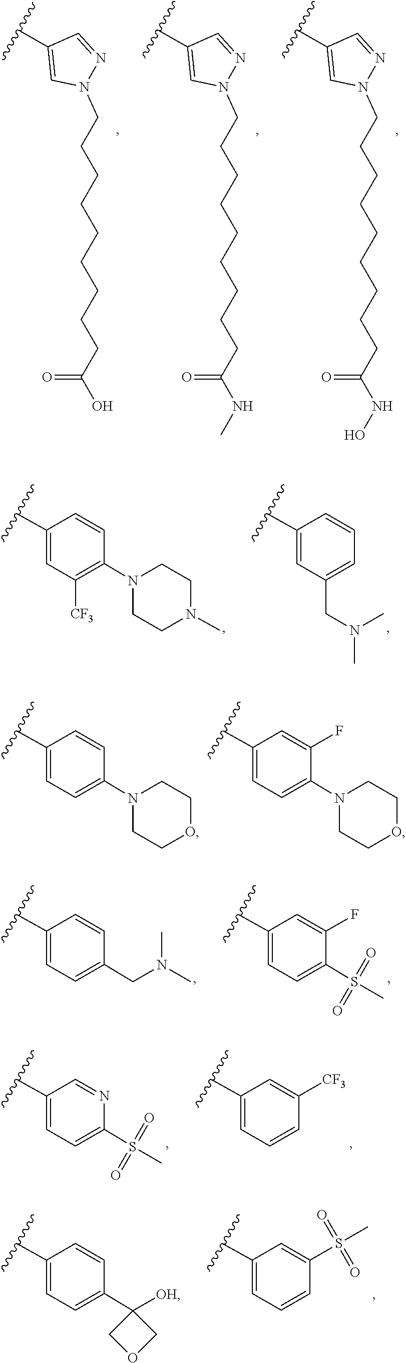

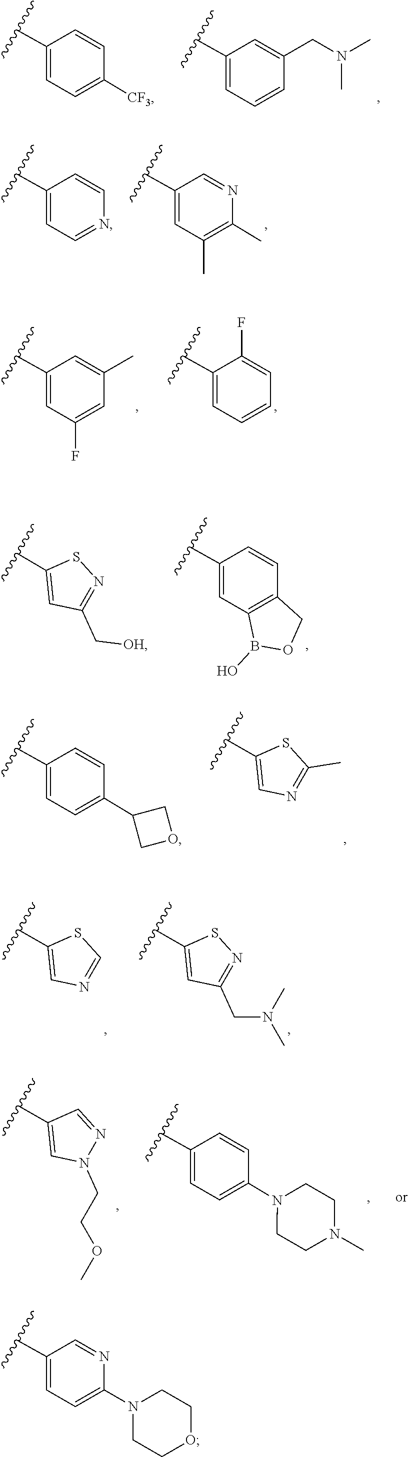

D) R³ is CN, OH, NH₂,

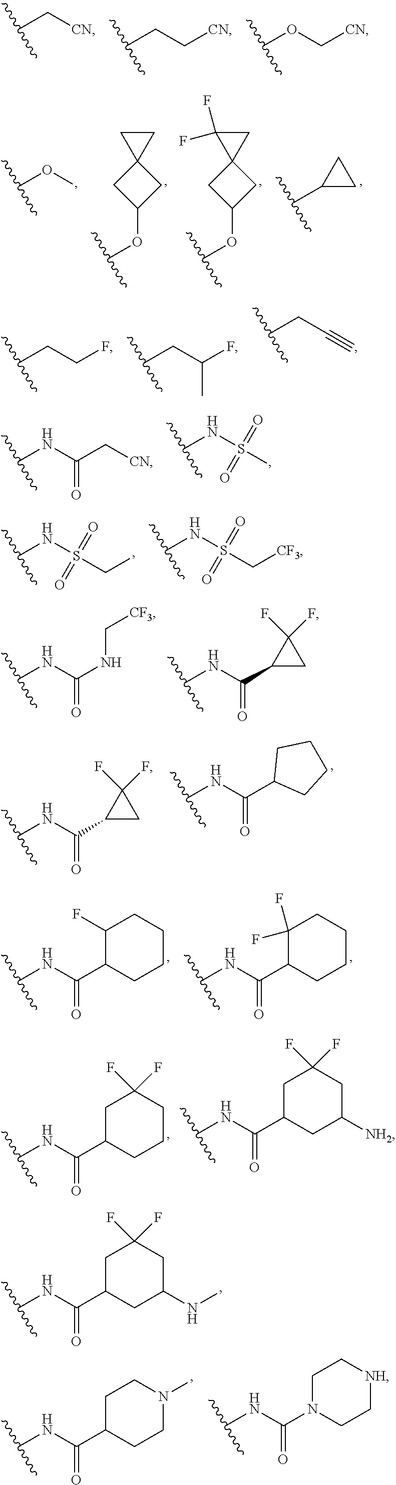

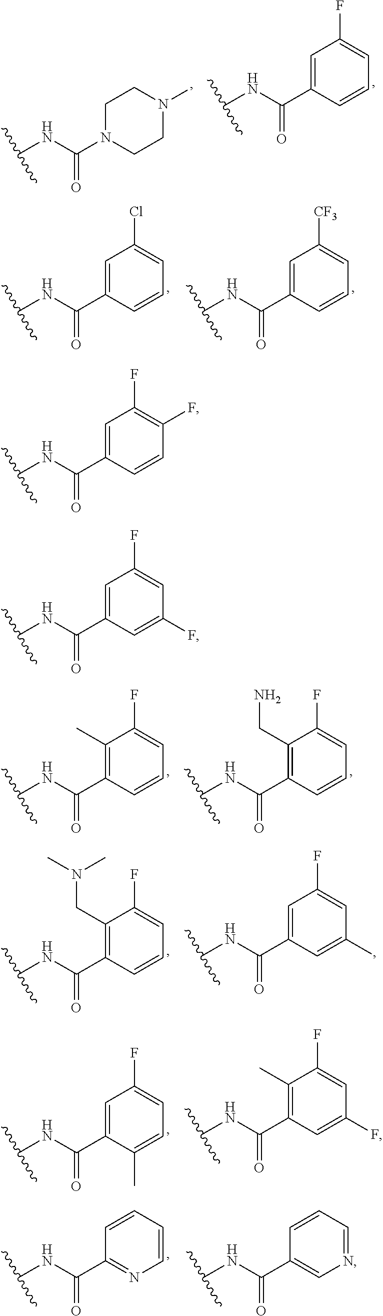

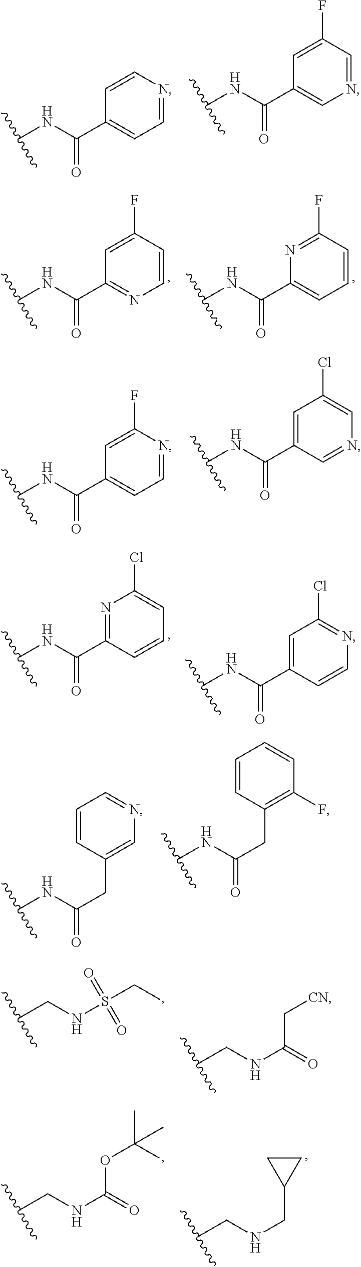

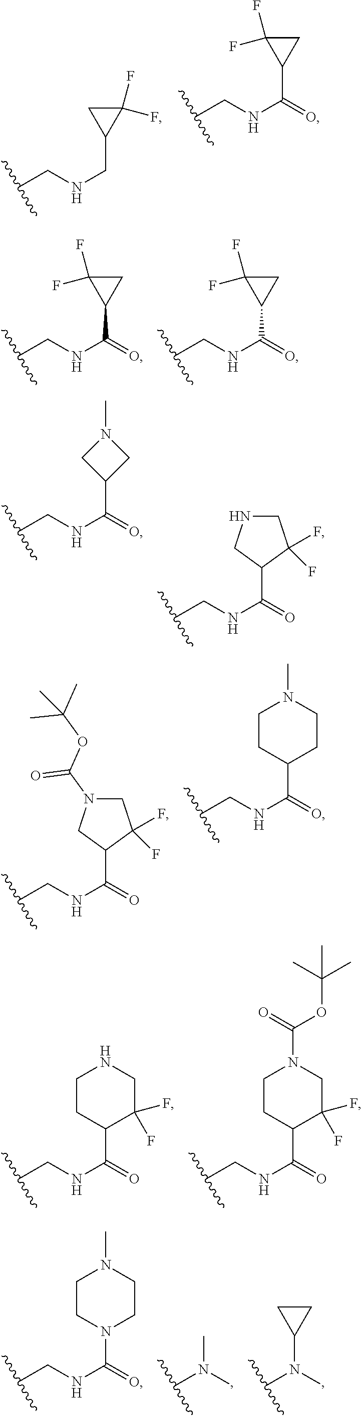

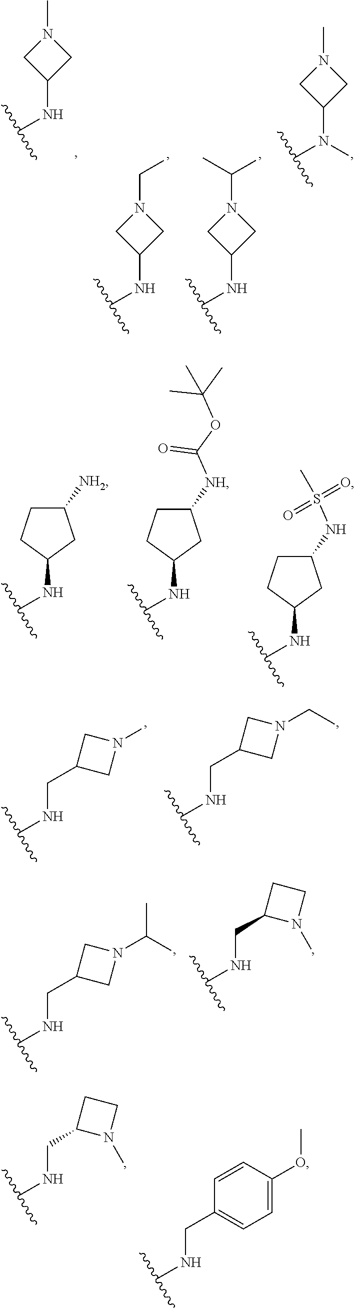

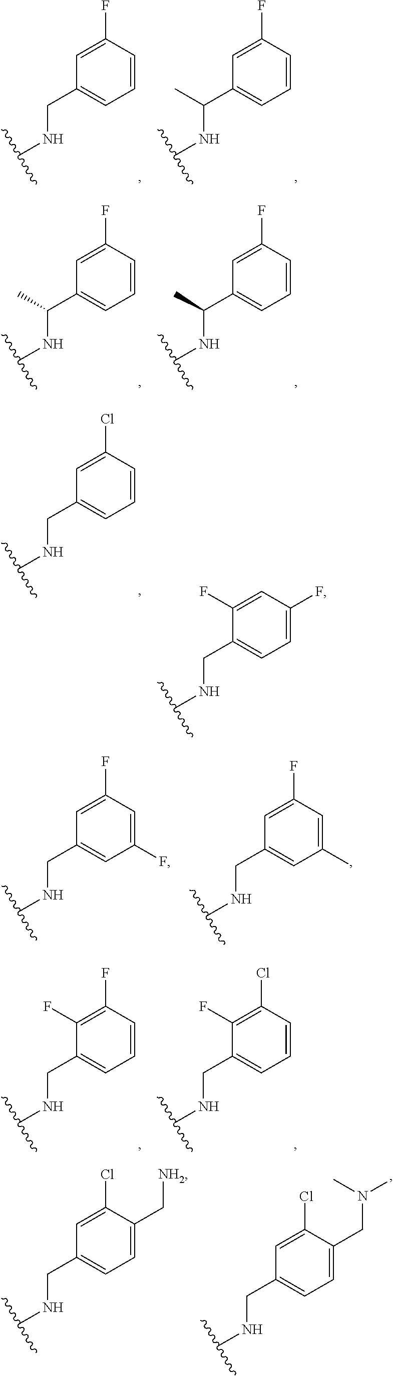

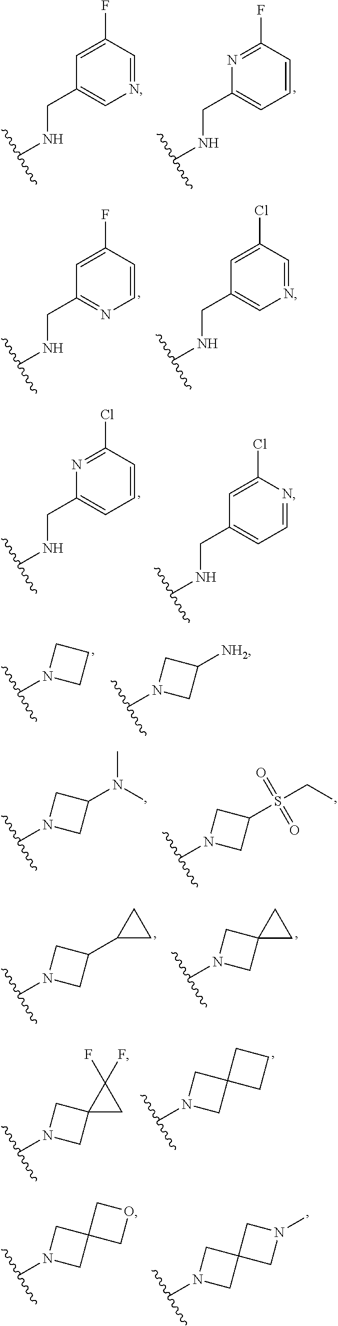

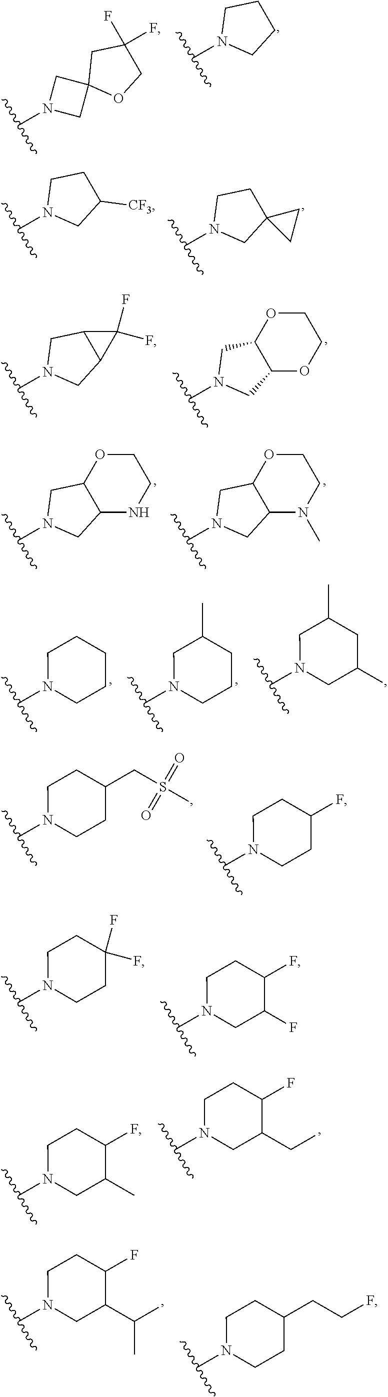

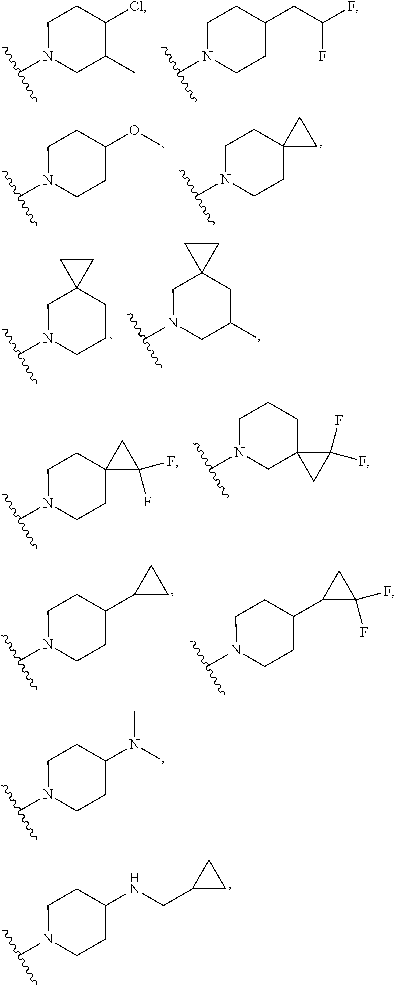

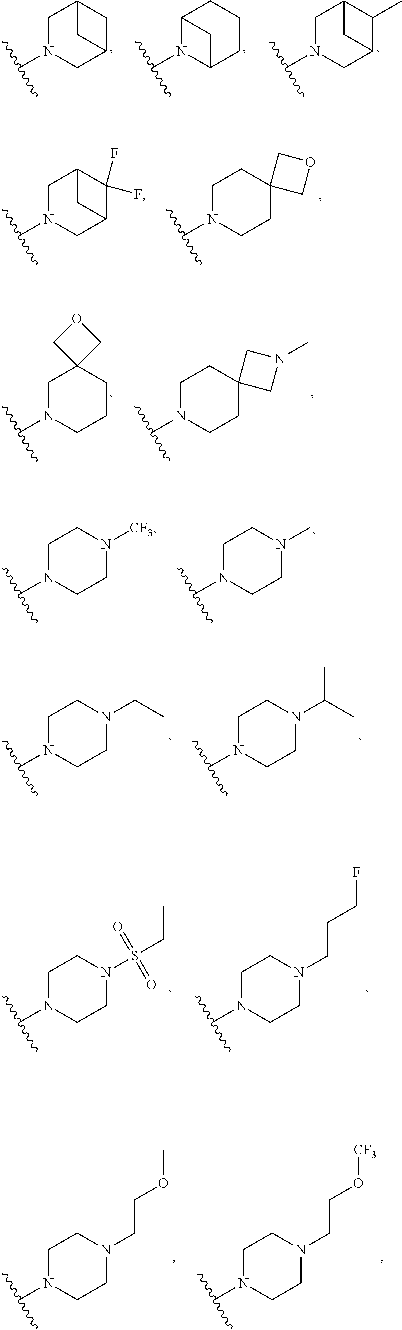

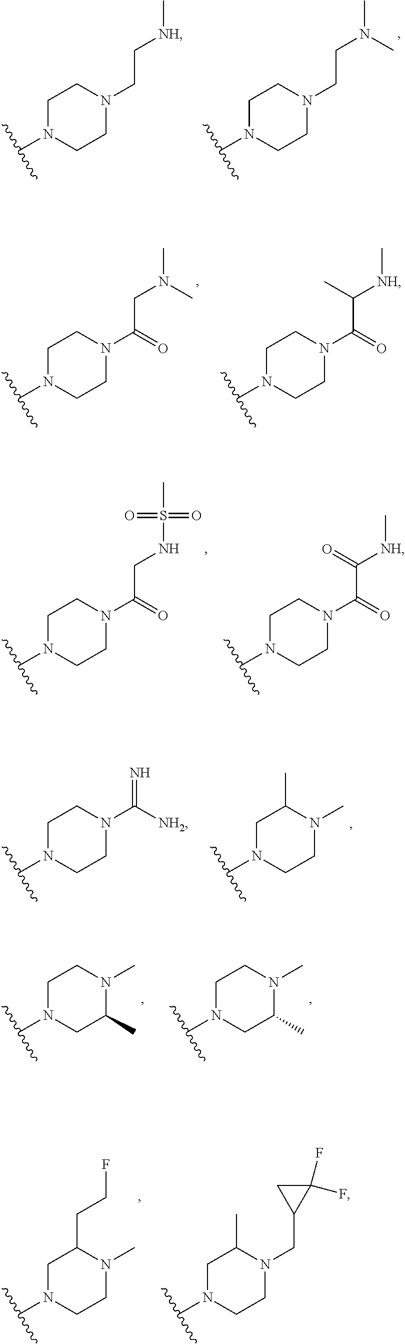

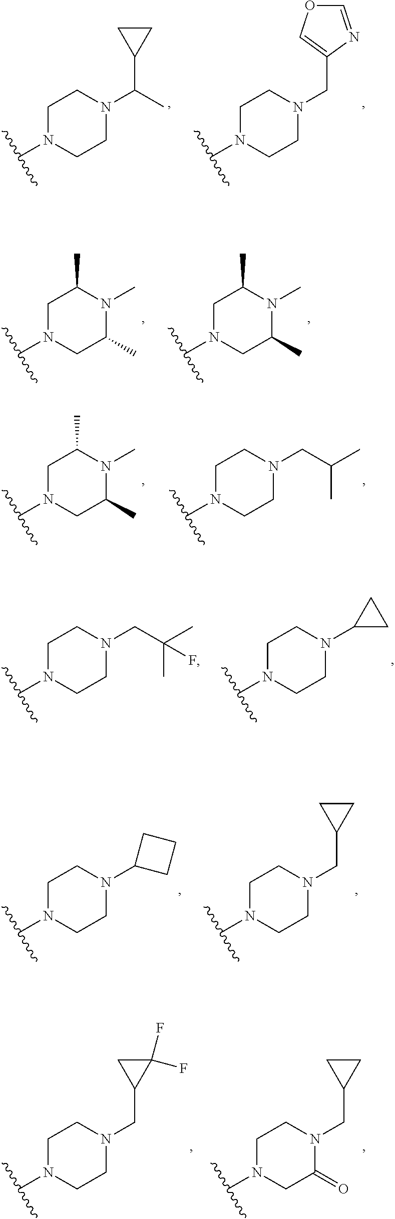

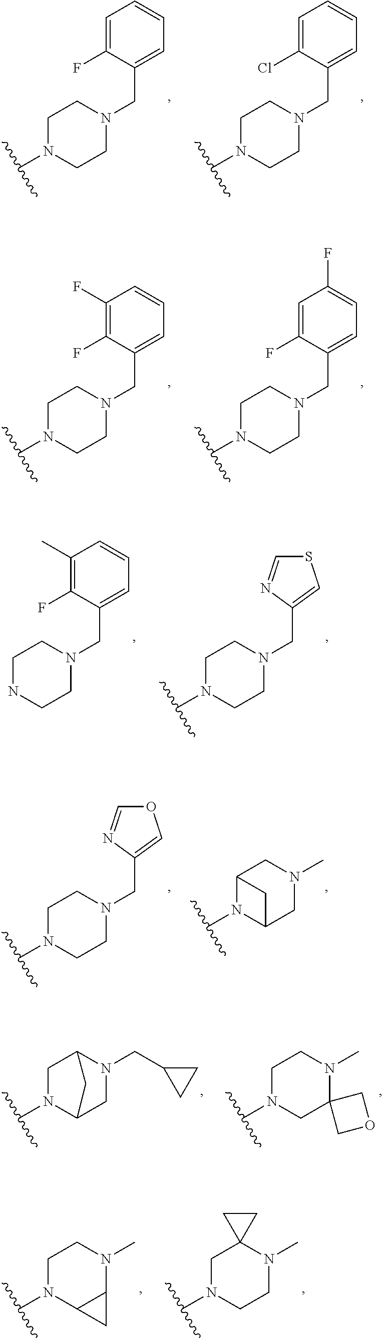

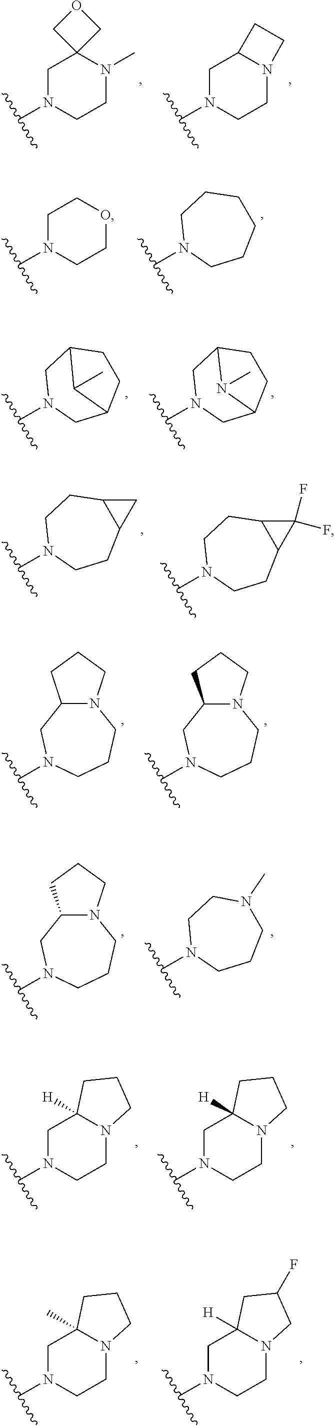

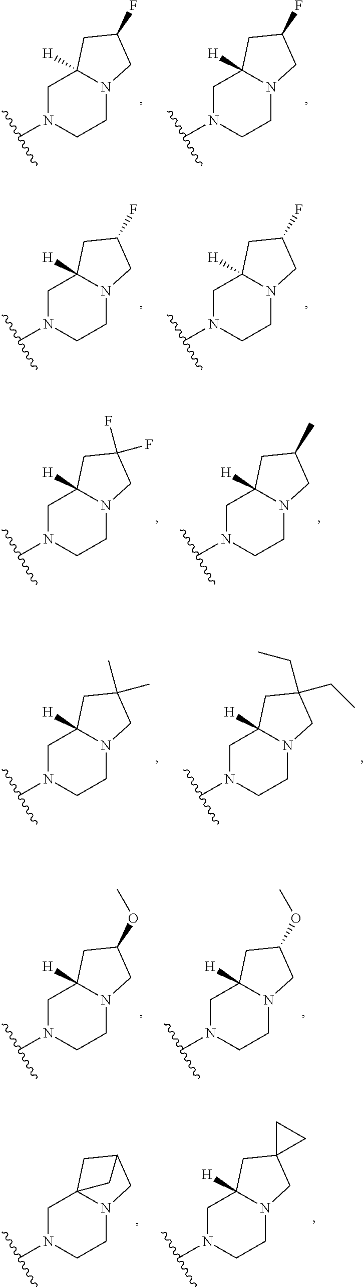

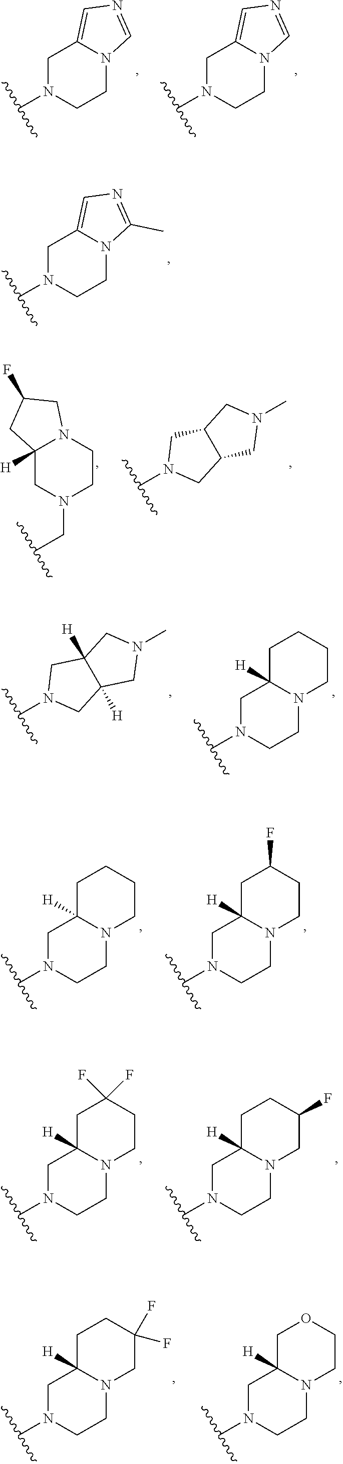

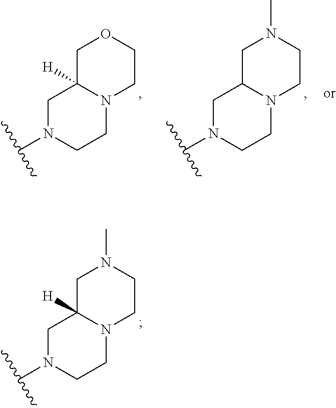 [SMILES: *N1CCN2CCOC[C@@H]2C1 ; *N1CCN2CCN(C)CC2C1 ; *N1CCN2CCN(C)C[C@H]2C1]

E) R⁴ is H, CH₃, CH₂CH₃, isopropyl, cyclopropyl, cyclobutyl, cyclopentyl, cyclohexyl, cycloheptyl, OH, CH₂OH, CH₂CH₂OH, CH₂F, CH₂CHF₂, CH₂CH₂F, CH₂CH₂Cl, CH₂-cyclopropyl, CH₂CN, CH₂CH₂CN, 3-fluorocyclobut-1-yl, tetrahydro-2H-pyran-4-yl, CH₂C(O)OCH₃, or CH₂OC(O)CH₃; or
F) R³ and R⁴, together with the atoms to which they are attached, combine to form

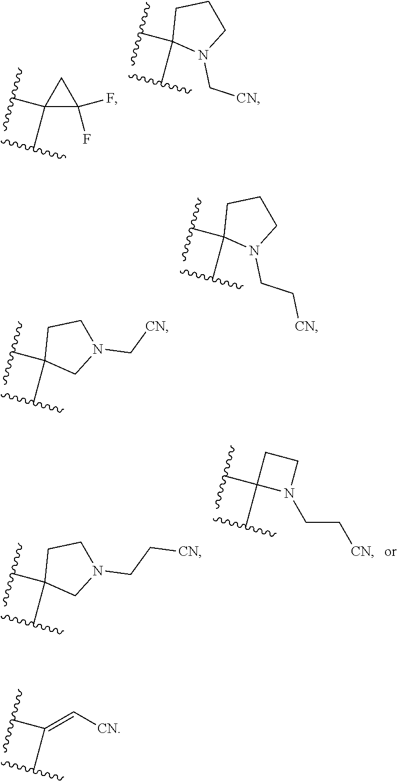

In some embodiments, the compounds provided herein are selected from:

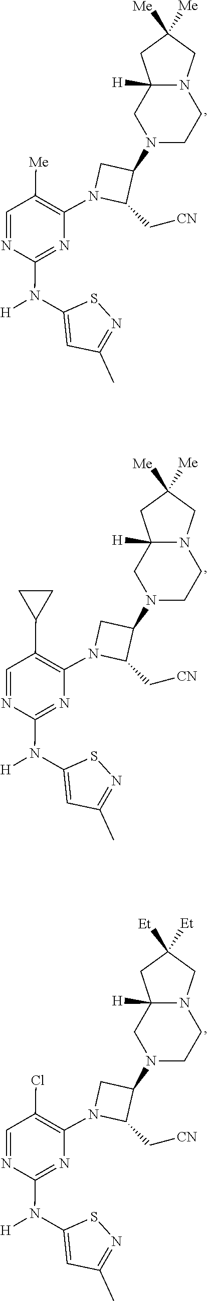

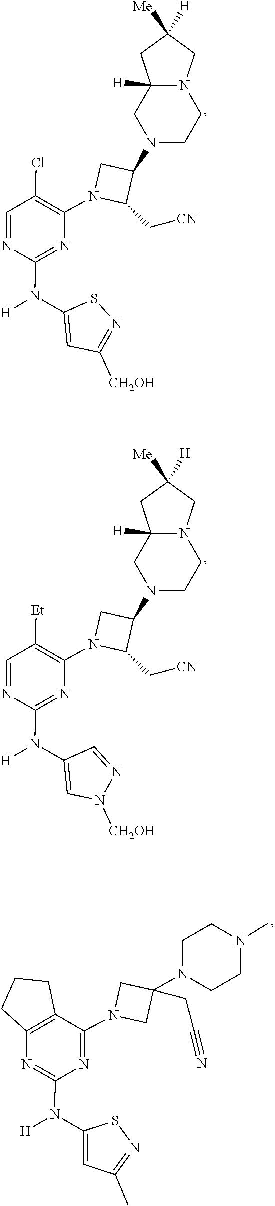 [SMILES: C[C@@H]1C[C@H]2CN([C@@H]3CN(c4nc(Nc5cc(CO)ns5)ncc4Cl)[C@H]3CC#N)CCN2C1 ; CCc1cnc(Nc2cnn(CO)c2)nc1N1C[C@@H](N2CCN3C[C@H](C)C[C@H]3C2)[C@@H]1CC#N ; Cc1cc(Nc2nc3c(c(N4CC(CC#N)(N5CCN(C)CC5)C4)n2)CCC3)sn1]

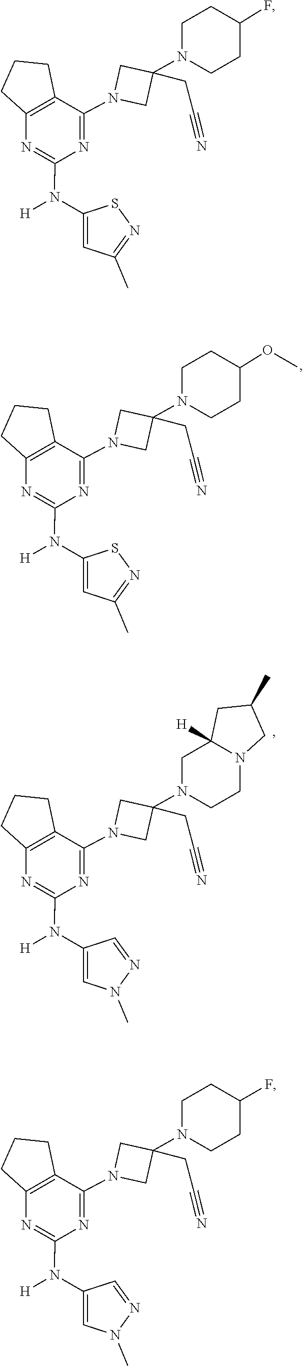 [SMILES: Cc1cc(Nc2nc3c(c(N4CC(CC#N)(N5CCC(F)CC5)C4)n2)CCC3)sn1 ; COC1CCN(C2(CC#N)CN(c3nc(Nc4cc(C)ns4)nc4c3CCC4)C2)CC1 ; C[C@@H]1C[C@H]2CN(C3(CC#N)CN(c4nc(Nc5cnn(C)c5)nc5c4CCC5)C3)CCN2C1 ; Cn1cc(Nc2nc3c(c(N4CC(CC#N)(N5CCC(F)CC5)C4)n2)CCC3)cn1]

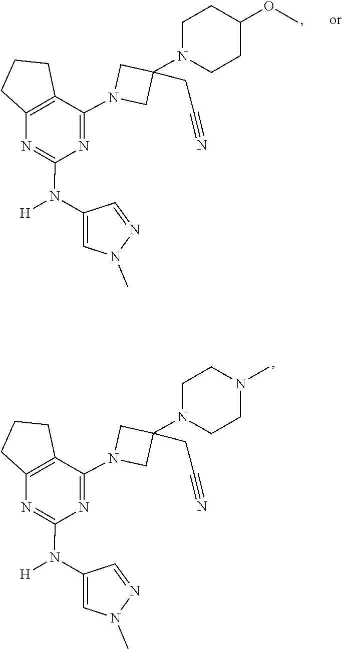 [SMILES: COC1CCN(C2(CC#N)CN(c3nc(Nc4cnn(C)c4)nc4c3CCC4)C2)CC1 ; CN1CCN(C2(CC#N)CN(c3nc(Nc4cnn(C)c4)nc4c3CCC4)C2)CC1]

or a pharmaceutically acceptable salt thereof.

Compounds described herein also include isotopically-labeled compounds wherein one or more atoms is replaced by an atom having the same atomic number, but an atomic mass or mass number different from the atomic mass or mass number predominantly found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In some embodiments, isotopically-labeled compounds are useful in drug or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In some embodiments, the compounds described herein are labeled by other means, including, but not limited to, the use of chromophores or fluorescent moieties, bioluminescent labels, or chemiluminescent labels.

### Compositions

In some embodiments, provided herein are compositions, comprising a compound provided herein. In some embodiments, the composition is a pharmaceutical composition, further comprising a pharmaceutically acceptable carrier. In some embodiments, the carrier is a solvent or an inert stabilizer, or both. In some embodiments, the inert stabilizer provides a dehydrating effect to the composition, which may enable a longer shelf life stability of the compounds for storing the composition. In some embodiments, the compositions may further include an additional pharmaceutical agent.

### Methods

The compounds described herein have kinase modulatory activity. In some embodiments, the kinase modulatory activity includes inhibition of one or more JAK proteins. In some embodiments, the kinase modulatory activity includes inhibition of ROCK1 or ROCK 2, or both.

Thus, in some embodiments, the compounds described herein are useful in treating a JAK- or ROCK-related disease in a subject in need thereof. In some embodiments, provided herein are methods of treating a disease in a subject in need thereof, comprising administering to the subject a therapeutically effective amount of a compound described herein.

In some embodiments, provided herein are methods of treating an eye disease in a subject in need thereof, comprising administering a therapeutically effective amount of a compound provided herein to the subject. In some embodiments, the eye disease includes, but is not limited to, ocular inflammatory conditions such as non-infectious uveitis, chorioretinitis, iritis, sterile conjunctivitis, keratitis, episcleritis, dry eye diseases, meibomian gland dysfunction, allergic conjunctivitis, injury-related ocular inflammation or dry eye syndrome, Primary or Secondary Sjögren's syndrome, redness, blepharitis, keratoconjunctivitis sicca, ocular hyperemia, macular degeneration (wet or dry), diabetic retinopathy, diabetic macular edema, retinal vein occlusion, age-related macular degeneration, geographic atrophy, posterior uveitis, retinal inflammation, Inflammation due to gene therapy vectors (e.g., viral vectors), graft versus host disease, blepharitis, Thygeson superficial punctate keratitis, or a combination thereof.

In some embodiments, provided herein are methods of treating a disease in a subject in need thereof, comprising administering a therapeutically effective amount of a compound provided herein to the subject, wherein the disease is selected from: neurodegenerative diseases or conditions such as Alzheimer's; ocular diseases, such as diabetic eye diseases, wet age-related macular degeneration, or dry age-related macular degeneration, inflammatory eye diseases, retinal degradation, and glaucoma; cardiovascular diseases; or cancer. Additional examples of diseases that may be treated by administration of the compounds provided herein include bone condition, obesity, hepatic disease, renal disease, pancreatitis, gastric disturbance, hypertension, fertility control, conditions of hair growth, nasal congestion, neurogenic bladder condition, gastrointestinal condition, dermatological condition, or respiratory indications.

In some embodiments, the JAK-related disease includes diseases and conditions involving the immune system including, for example, organ transplant rejection (e.g., allograft rejection and graft versus host disease). Further examples of JAK-related diseases or conditions include autoimmune diseases such as alopecia areata, alopecia universalis, polycythemia vera, vitiligo, multiple sclerosis, rheumatoid arthritis, juvenile arthritis, psoriatic arthritis, type I diabetes, lupus, psoriasis, inflammatory bowel disease, ulcerative colitis, Crohn's disease, myasthenia gravis, immunoglobulin nephropathies, myocarditis, autoimmune thyroid conditions, chronic obstructive pulmonary disease (COPD), and the like. In some embodiments, the autoimmune disease is arthritis.

Further examples of JAK-related diseases or conditions include allergic conditions such as asthma, food allergies, eczematous dermatitis, contact dermatitis, atopic dermatitis (atropic eczema), and rhinitis. Further examples of JAK-related diseases or conditions include viral diseases such as Epstein Barr Virus (EBV), Hepatitis B, Hepatitis C, HIV, HTLV 1, Varicella-Zoster Virus (VZV) and Human Papilloma Virus (HPV).

Further examples of JAK-related diseases or conditions include those characterized by solid tumors (e.g., prostate cancer, renal cancer, hepatic cancer, pancreatic cancer, gastric cancer, breast cancer, lung cancer, cancers of the head and neck, thyroid cancer, glioblastoma, Kaposi's sarcoma, Castleman's disease, uterine leiomyosarcoma, melanoma etc.), hematological cancers (e.g., lymphoma, leukemia such as acute lymphoblastic leukemia (ALL), acute myelogenous leukemia (AML) or multiple myeloma), and skin cancer such as cutaneous T-cell lymphoma (CTCL) and cutaneous B-cell lymphoma. Example CTCLs include Sezary syndrome and mycosis fungoides. Other examples of JAK-related diseases or conditions include pulmonary arterial hypertension.

Other examples of JAK-related diseases or conditions include inflammation-associated cancers. In some embodiments, the cancer is associated with inflammatory bowel disease. In some embodiments, the inflammatory bowel disease is ulcerative colitis. In some embodiments, the inflammatory bowel disease is Crohn's disease. In some embodiments, the inflammation-associated cancer is colitis-associated cancer. In some embodiments, the inflammation-associated cancer is colon cancer or colorectal cancer. In some embodiments, the cancer is gastric cancer, gastrointestinal carcinoid tumor, gastrointestinal stromal tumor (GIST), adenocarcinoma, small intestine cancer, hematological cancers, or rectal cancer.

In some embodiments, provided herein are methods of treating an ocular condition in a subject in need thereof, comprising administering to the subject a therapeutically effective amount of a compound, a composition, or a pharmaceutical composition provided herein.

In some embodiments, provided herein are methods of reducing ocular inflammation in a subject in need thereof, comprising administering to the subject a therapeutically effective amount of compound, a composition, or a pharmaceutical composition provided herein.

In some embodiments, provided herein are methods of treating an ocular condition in a subject in need thereof, comprising administering to the subject a therapeutically effective amount of a compound, or a pharmaceutically acceptable salt thereof, provided herein.

In some embodiments, the ocular disease or condition is dry eye.

In some embodiments, the ocular disease or condition is meibomian gland dysfunction (MGD).

In some embodiments, the ocular disease or condition is uveitis.

In some embodiments, the ocular disease or condition is blepharitis.

In another aspect, provided herein are methods of reducing inflammation in a subject in need thereof, comprising administering to the subject a therapeutically effective amount of a compound, or a pharmaceutically acceptable salt thereof, provided herein.

In some embodiments of these aspects, a compound or pharmaceutical composition is administered topically to an eye of the subject.

In some embodiments, provided herein are methods of treating an ocular disease or condition in a subject in need thereof, comprising administering to the subject a therapeutically effective amount of a compound provided herein.

In some embodiments, provided herein are methods of treating an ocular disease or condition in a subject in need thereof, comprising administering to the subject a therapeutically effective amount of a compound provided herein.

In some embodiments, provided herein are methods of reducing the signs or symptoms of dry eye disease (DED) or meibomian gland dysfunction (MGD) in a subject in need thereof, comprising administering to the subject a therapeutically effective amount of a compound provided herein.

In some embodiments, provided herein are methods of reducing intraocular pressure in a subject in need thereof, comprising administering to the subject a therapeutically effective amount of a compound provided herein.

In some embodiments of the methods provided herein, the methods further comprise administering a therapeutically effective amount of one or more additional therapeutic agents. In some embodiments, the one or more additional therapeutic agents is a beta blocker (e.g., levobunalol, timolol, betaxolol, or metipranolol), an alpha-agonist (e.g., apraclonidine or brimonidine), a carbonic anhydrase inhibitor (e.g., dorzolamide or brinzolamide), a prostaglandin, a prostaglandin-like compound (e.g., AR-102, latanoprost, bimatoprost, tafluprost, or travoprost), a miotic or cholinergic agent (e.g., pilocarpine or carbachol), an epinephrine or nor-epinephrin compound (e.g., dipivefrin), a neuroprotective or anti-inflammatory compound (e.g., alfibercept), or a corticosteroid (e.g., dexamethasone). In some embodiments, the one or more additional therapeutic agents is a prostaglandin or a prostaglandin-like compound. In some embodiment, the prostaglandin-like compound is AR-102, latanoprost, bimatoprost, tafluprost, or travoprost. In another embodiment, the additional therapeutic agents are other JAK inhibitors or corticosteroids. As explained above, the administration of a compound of the present disclosure can be concomitantly, as a mixture in a single pharmaceutical composition, or chemically conjugated directly to each other or through a linker.

Also provided herein are methods of treating an autoimmune disease or condition in a subject in need thereof, comprising administering to the subject a therapeutically effective amount of compound, a composition, or a pharmaceutical composition provided herein.

In some embodiments, provided herein are methods of treating an autoimmune disease or condition in a subject in need thereof, comprising administering to the subject a therapeutically effective amount of a compound provided herein.

In some embodiments, provided herein are methods of treating an autoimmune disease or condition in a subject in need thereof, comprising administering to the subject a therapeutically effective amount of a compound provided herein.

In some embodiments, the autoimmune disease or condition is multiple sclerosis, rheumatoid arthritis, juvenile arthritis, psoriatic arthritis, type I diabetes, lupus, psoriasis, inflammatory bowel disease, ulcerative colitis, Crohn's disease, myasthenia gravis, immunoglobulin nephropathies, myocarditis, autoimmune thyroid conditions, or chronic obstructive pulmonary disease.

In some embodiments, the subject comprises a refractory disease. In some embodiments, the refractory disease comprises a refractory cancer.

The actual route of administration of a compound, a composition, or a combination disclosed herein used can be determined by a person of ordinary skill in the art by taking into account factors, including, without limitation, the type of JAK- or ROCK-related disease, the location of the JAK- or ROCK-related disease, the cause of the JAK- or ROCK-related disease, the severity of the JAK- or ROCK-related disease, the duration of treatment desired, the degree of relief desired, the duration of relief desired, the particular compound, composition, or combination, the rate of excretion of the compound, composition, or combination used, the pharmacodynamics of the compound, composition, or combination used, the nature of the other compounds to be included in the composition or combination, the particular route of administration, the particular characteristics, history and risk factors of the individual, such as, e.g., age, weight, general health and the like, the response of the individual to the treatment, or any combination thereof. An effective dosage amount of a compound, a composition, or a combination disclosed herein can thus readily be determined by the person of ordinary skill in the art considering all criteria and utilizing his best judgment on the individual's behalf.

### Kits

In some embodiments, provided herein are packaged compounds, packaged compositions, or packaged pharmaceutical compositions, comprising a container holding a therapeutically effective amount of a compound described herein, and instructions for using the compound in accordance with one or more of the methods provided herein.

The present compounds and associated materials can be finished as a commercial product by the usual steps performed in the present field, for example by appropriate sterilization and packaging steps. For example, the material can be treated by UV/vis irradiation (200-500 nm), for example using photo-initiators with different absorption wavelengths (e.g. Irgacure 184, 2959), preferably water-soluble initiators (e.g. Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (e.g. by the addition of specific product information leaflets) into suitable containers (boxes, etc.).

According to further embodiments, the present compounds can also be provided in kit form combined with other components necessary for administration of the material to the patient. For example, disclosed kits, such as for use in the treatment of cancer, can further comprise, for example, administration materials.

The kits are designed in various forms based on the specific deficiencies they are designed to treat.

The compounds or compositions provided herein may be prepared and placed in a container for storage at ambient or elevated temperature. When the compound or composition is stored in a polyolefin plastic container as compared to a polyvinyl chloride plastic container, discoloration of the compound or composition may be reduced, whether dissolved or suspended in a liquid composition (e.g., an aqueous or organic liquid solution), or as a solid. Without wishing to be bound by theory, the container may reduce exposure of the container's contents to electromagnetic radiation, whether visible light (e.g., having a wavelength of about 380-780 nm) or ultraviolet (UV) light (e.g., having a wavelength of about 190-320 nm (UV B light) or about 320-380 nm (UV A light)). Some containers also include the capacity to reduce adherence or adsorption of the active agent to the surface of the container. Some containers also include the capacity to reduce exposure of the container's contents to infrared light, or a second component with such a capacity. The containers that may be used include those made from a polyolefin such as polyethylene, polypropylene, polyethylene terephthalate, polycarbonate, polymethylpentene, polybutene, or a combination thereof, especially polyethylene, polypropylene, or a combination thereof. In some embodiments, the container is a glass container. The container may further be disposed within a second container, for example, a paper, cardboard, paperboard, metallic film, or foil, or a combination thereof, container to further reduce exposure of the container's contents to UV, visible, or infrared light. Compounds and compositions benefiting from reduced discoloration, decomposition, or both during storage, include eye drop solutions or implants that include a compound or composition thereof provided herein. The compounds or compositions provided herein may need storage lasting up to, or longer than, three months; in some cases up to, or longer than one year. The containers may be in any form suitable to contain the contents; for example, a bag, a bottle, or a box.

The following examples further illustrate aspects of the present disclosure. However, they are in no way a limitation of the teachings or disclosure as described herein.

### EXAMPLES

### Example 1: ROCK and JAK Assays

ROCK Kinase Inhibition Assays. All compounds were initially prepared as 10 mM stocks in anhydrous dimethylsulfoxide (DMSO). A 20 μL aliquot of the 10 mM solutions was transferred to individual wells in column 1 of a 96-well polypropylene microtiter plate (Corning #3363) and diluted with DMSO to give a final compound concentration of 4 mM. Test compounds were then serially diluted 1:5 in DMSO for an 11-point concentration response and further diluted in the assay buffer bringing all compound concentrations to a final range of 100 μM to 10 μM in 2.5% DMSO. The assay was performed in white 96-well, flat-bottom, half-area, non-binding assay plate (Corning #3642) in assay buffer consisting of 20 mM HEPES (pH 7.5), 10 mM MgCl₂*6H₂O, 100 μM sodium orthovanadate, 0.05% CHAPS and 0.1% bovine serum albumin. A 10 μL aliquot of compound from each well of the intermediate dilution plate and 20 μL of a 2× substrate/enzyme solution containing acceptor substrate (800 nM RSK2 peptide KRRRLSSLRA (SEQ ID NO:1)), ROCK2 enzyme (10 nM), or ROCK1 enzyme, and 1,4-Dithiothreitol (DTT, 2 uM) were added to all wells. The reaction was initiated by the addition of 10 μL of 4× stock solution ATP (2 μM). Reactions were thoroughly mixed manually, covered, and allowed to incubate at room temperature for 75 min. Protein kinase activity was quantitated using Promega's KINASE-GLO™ luminescent Kinase Assay Kit according to the manufacturer's directions. ATP concentrations remaining in Test wells following the termination of the enzymatic reaction were compared against control wells containing equivalent amounts of DMSO containing no inhibitor (CTRL). ATP concentrations in both Test wells and CTRL wells were normalized against background (BKG) ATP concentrations in wells containing concentrations of inhibitor that completely inhibited the protein kinase under investigation (i.e. a concentration that prevented any consumption of ATP over the course of the incubation). Percent of Control (POC) values were determined for each concentration of compound tested according to the equation:

POC=((Test well value−BKG)/(CTRL−BKG))*100

IC50 values were calculated using the following 4-parameter logistic curve-fitting algorithm:

f(x)=(A+((B−A)/(1+((x/C)\hat{ }D))))

IC50 values were converted to Ki values using the following Cheng-Prusoff Equation:

Kᵢ=IC₅₀/(1+([ATP]/Km ATP])).

JAK Kinase Assays. Compounds were prepared in the same manner as described in the ROCK Kinase Assay with the exception to the substrate and enzyme. The JAK 2X substrate/enzyme solution consisted of acceptor substrate (800 nM Abl peptide EAIYAAPFAKKK (SEQ ID NO:2)), JAK1, TYK2, JAK2 or JAK3 enzyme (10 nM) and DTT (2 uM). All other steps and solutions remain identical to the ROCK Kinase Assay above.

### Example 2. Topical Ocular Composition

Topical ocular pharmaceutical compositions for treating inflammation are prepared by conventional methods and formulated as shown in Table 1.

TABLE 1

| Ingredient | Amount (wt %) |
|---|---|
| Active agent (compound provided herein) | 0.30 |
| Citric Acid | 0.1 |
| Hydroxypropyl methylcellulose | 0.3 |
| Sodium Chloride | 0.77 |
| Potassium chloride | 0.12 |
| Disodium EDTA | 0.05 |
| Benzalkonium chloride | 0.01 |
| HCl and/or NaOH | pH 4.5-6.5 |
| Purified water | q.s. to 100% |

When the composition is topically administered to one or both eyes once daily, the above composition decreases ocular inflammation in a subject suffering from Meibomian Gland Dysfunction (MGD) or Dry Eye Disease (DED).

### Example 3. Topical Ocular Composition

Topical ocular pharmaceutical compositions for treating inflammation are prepared by conventional methods and formulated as shown in Table 2.

TABLE 2

| Ingredient | Amount (wt %) |
|---|---|
| Active agent (compound provided herein) | 0.30 |
| Citric Acid | 0.067 |
| Sodium Citrate Dihydrate | 0.48 |
| Sodium Chloride | 0.70 |
| HCl and/or NaOH | pH 6.0 |
| Purified water | q.s. to 100% |

When the composition is topically administered to one or both eyes once daily, the above composition decreases ocular inflammation in a subject suffering from meibomian gland dysfunction (MGD) or dry eye disease (DED).

### Example 4. Pharmacological Activity for Glaucoma Assay

Pharmacological activity for glaucoma can be demonstrated using assays designed to test the ability of the subject compounds to decrease intraocular pressure. Examples of such assays are described in the following reference, incorporated herein by reference: C. Liljebris, G. Selen, B. Resul, J. Stjernschantz, and U. Hacksell, “Derivatives of 17-phenyl-18, 19, 20-trinorprostaglandin F2a Isopropyl Ester: Potential Anti-glaucoma Agents”, Journal of Medicinal Chemistry 1995, 38 (2): 289-304.

### Example 5. General Synthesis

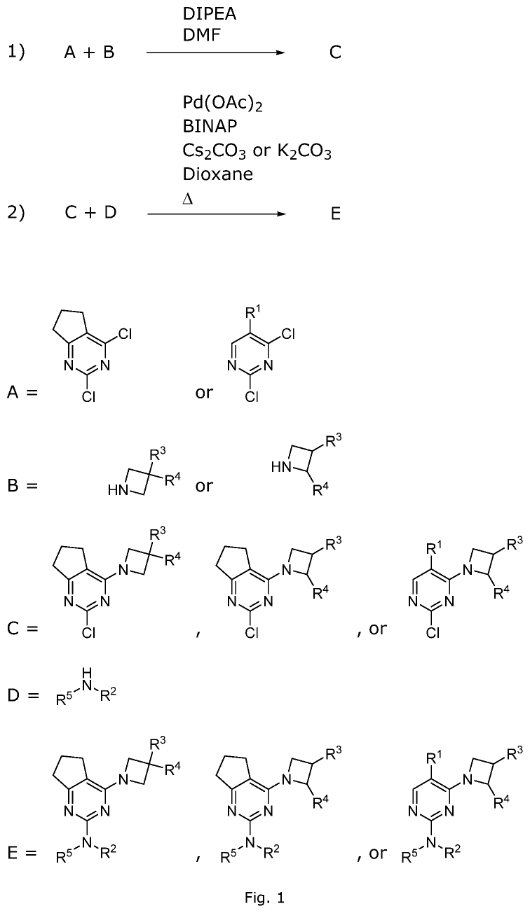

The compounds provided herein may be synthesized by a number of methods, including the synthetic scheme shown in FIG. 1.

While the disclosure has been described in detail and with reference to specific embodiments thereof, it will be apparent to one skilled in the art that various changes and modifications can be made without departing from the spirit and scope of the disclosure.

Groupings of alternative elements or embodiments disclosed herein may be referred to and claimed individually or in any combination with other members of the group or other elements found herein.

The contents of all references (including literature references, issued patents, published patent applications, and co-pending patent applications) cited throughout this application are hereby expressly incorporated herein in their entireties. Unless otherwise defined, all technical and scientific terms used herein are accorded the meaning commonly known to one with ordinary skill in the art.

Those skilled in the art will recognize, or be able to ascertain, using no more than routine experimentation, many equivalents of the specific embodiments of the invention described herein. Such equivalents are intended to be encompassed by the following claims.

1. A compound, having a formula: 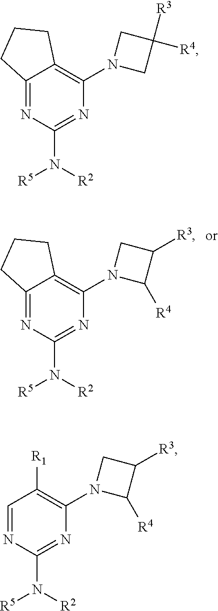
or a pharmaceutically acceptable salt thereof;
wherein
R⁵ is H or S(O)₂—(C₁₋₆ alkyl);
R¹ is H, CN, halogen, C₁₋₆ alkyl, O—(C₁₋₆ alkyl), or C₃₋₇ cycloalkyl;
R² is C₆₋₁₆ aryl or C₂₋₁₅ heterocyclyl, each of which may be substituted with 1 or 2 groups selected, independently, from OH, halogen, C₁₋₆ alkyl, (C₁₋₆ alkylene)-OH, O—(C₁₋₆ alkyl), (C₁₋₆ alkylene)-O—(C₁₋₆ alkyl), S(O)₂—(C₁₋₆ alkyl), C₃₋₇ cycloalkyl, (C₁₋₁₆ alkylene)-C(O)OH, (C₁₋₁₆ alkylene)-C(O)N(H)—(C₁₋₆ alkyl), (C₁₋₁₆ alkylene)-C(O)N(H)—OH, or (C₁₋₆ alkylene)-N(C₁₋₆ alkyl)-(C₁₋₆ alkyl);
R³ is CN, OH, NH₂, C₁₋₆ alkyl, C₁₋₆ alkynyl, C₃₋₇ cycloalkyl, (C₁₋₃ alkylene)-CN, (C₁₋₃ alkylene)-NH₂, (C₁₋₃ alkylene)-N(H)C(O)—(C₃₋₇ cycloalkyl), (C₁₋₃ alkylene)-N(H)C(O)—(C₂₋₈ heterocycloalkyl), O—(C₃₋₇ cycloalkyl), O—(C₁₋₃ alkylene)-CN, N(H)C(O)—(C₃₋₇ cycloalkyl), N(H)C(O)—(C₂₋₈ heterocycloalkyl), N(H)C(O)—(C₂₋₅ heteroaryl), N(H)C(O)—(C₁₋₃ alkylene)-(C₂₋₅ heteroaryl), N(H)C(O)—(C₆₋₁₀ aryl), N(H)C(O)—(C₁₋₃ alkylene)-(C₆₋₁₀ aryl), N(H)C(O)NH₂, N(H)(C₁₋₆ alkyl), N(H)(C₃₋₇ cycloalkyl), N(C₁₋₆ alkyl)(C₃₋₇ cycloalkyl), N(H)(C₁₋₃ alkylene)-(C₂₋₈ heterocycloalkyl), N(H)(C₁₋₃ alkylene)-(C₂₋₅ heteroaryl), N(H)(C₁₋₃ alkylene)-(C₆₋₁₀ aryl), or C₂₋₁₅ heterocyclyl, each of which may be substituted with 1, 2, 3, or 4 groups selected, independently, from halogen, O, OH, O—(C₁₋₆ alkyl), S(O)₂—(C₁₋₆ alkyl), S(O)₂—(C₁₋₆ haloalkyl), C(NH₂)(NH), C₁₋₆ alkyl, C₁₋₆ haloalkyl, (C₁₋₆ alkylene)-OH, (C₁₋₆ alkylene)-O—(C₁₋₆ alkyl), (C₁₋₆ alkylene)-O—(C₁₋₆ haloalkyl), (C₁₋₆ alkylene)-S(O)₂—(C₁₋₆ alkyl), (C₁₋₆ alkylene)-NH₂, (C₁₋₆ alkylene)-N(H)(C₁₋₆ alkyl), (C₁₋₆ alkylene)-N(C₁₋₆ alkyl)(C₁₋₆ alkyl), (C₁₋₆ alkylene)-(C₃₋₇ cycloalkyl), (C₁₋₆ alkylene)-(C₃₋₇ halocycloalkyl), (C₁₋₆ alkylene)-(C₂₋₅ heteroaryl), (C₁₋₆ alkylene)-(C₆₋₁₀ haloaryl), NH₂, N(H)(C₁₋₆ alkyl), N(C₁₋₆ alkyl)(C₁₋₆ alkyl), N(H)—(C₁₋₃ alkylene)-(C₃₋₇ cycloalkyl), N(C₁₋₆ alkyl)-(C₁₋₃ alkylene)-(C₃₋₇ cycloalkyl), N(H)C(O)O—(C₁₋₆ alkyl), N(H)S(O)₂—(C₁₋₆ alkyl), C(O)—(C₁₋₆ alkyl), C(O)—(C₁₋₆ alkylene)-CN, C(O)—(C₁₋₆ alkylene)-NH₂, C(O)—(C₁₋₆ alkylene)-N(H)(C₁₋₆ alkyl), C(O)—(C₁₋₆ alkylene)-N(C₁₋₆ alkyl)(C₁₋₆ alkyl), C(O)—(C₁₋₆ alkylene)-N(H)[S(O)₂—C₁₋₆ alkyl], C(O)—(C₁₋₆ alkylene)-N(C₁₋₆ alkyl)[S(O)₂—C₁₋₆ alkyl], or C(O)O—(C₁₋₆ alkyl);
R³ is C₂₋₈ heterocycloalkyl, or C₂₋₈ heterocycloalkyl substituted with 1 or 2 groups selected, independently, from C₁₋₆ alkyl, F, Cl, Br, or O(C₁₋₆ alkyl); and
R⁴ is H, OH, C₁₋₆ alkyl, (C₁₋₆ alkylene)-OH, C₁₋₆ haloalkyl, C₃₋₇ cycloalkyl, C₃₋₇ halocycloalkyl, (C₁₋₃ alkylene)-(C₃₋₇ cycloalkyl), (C₁₋₆ alkylene)CN, (C₁₋₆ alkylene)-C(O)O—(C₁₋₆ alkyl), (C₁₋₆ alkylene)-OC(O)—(C₁₋₆ alkyl), or C₂₋₈ heterocycloalkyl;
or R³ and R⁴, together with the atoms to which they are attached, combine to form C₃₋₇ cycloalkyl, C₂₋₈ heterocycloalkyl, CC(H)—(C₀₋₆ alkylene)CN, C₃₋₇ cycloalkyl substituted with 1 or 2 groups selected, independently, from halogen or (C₁₋₆ alkylene)CN, or C₂₋₈ heterocycloalkyl substituted with 1 or 2 groups selected, independently, from halogen or (Ci-6 alkylene)CN.

2. The compound of claim 1, having a formula: 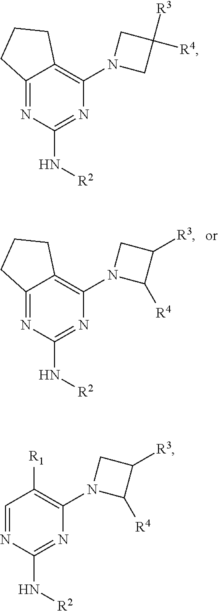
or a pharmaceutically acceptable salt thereof;
wherein
R¹ is H, F, Cl, Br, C₁₋₆ alkyl, or C₃₋₇ cycloalkyl;
R² is C₂₋₅ heteroaryl, or C₂₋₅ heteroaryl substituted with 1 or 2 groups selected, independently, from C₁₋₆ alkyl or (C₁₋₆ alkylene)OH;
R³ is C₂₋₈ heterocycloalkyl, or C₂₋₈ heterocycloalkyl substituted with 1 or 2 groups selected, independently, from C₁₋₆ alkyl, F, Cl, Br, or O(C₁₋₆ alkyl); and
R⁴ is (C₁₋₃ alkylene)CN.

3. The compound of claim 1, having a formula: 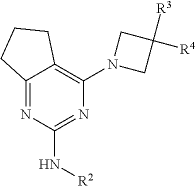
or a pharmaceutically acceptable salt thereof.

4. The compound of claim 1, having a formula: 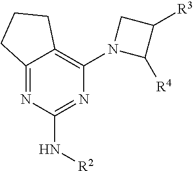
or a pharmaceutically acceptable salt thereof.

5. The compound of claim 1, having a formula: 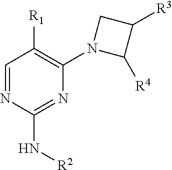
or a pharmaceutically acceptable salt thereof.

6. The compound of claim 1, wherein R¹ is F, Cl, Br, C₁₋₃ alkyl, or C₃₋₄ cycloalkyl.

7. The compound of claim 1, wherein R² is C₃₋₄ heteroaryl, or C₃₋₄ heteroaryl substituted with C₁₋₃ alkyl or (C₁₋₃ alkylene)OH.

8. The compound of claim 1, wherein R³ is C₄₋₈ heterocycloalkyl, or C₄₋₈ heterocycloalkyl substituted with 1 or 2 groups selected, independently, from C₁₋₃ alkyl, F, Cl, Br, or O(C₁₋₃ alkyl).

9. The compound of claim 1, wherein R⁴ is CH₂CN.

10. The compound of claim 2, having a formula: 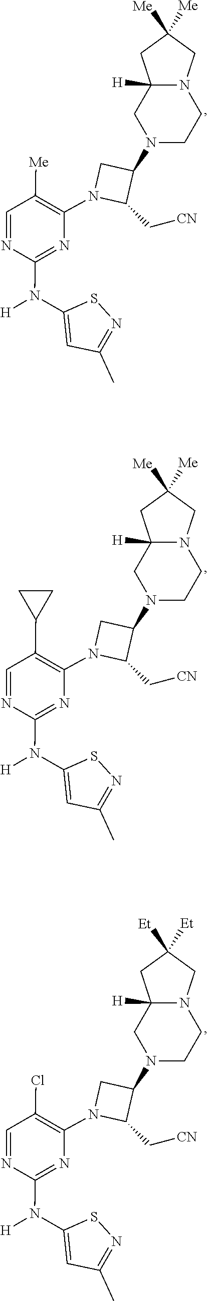 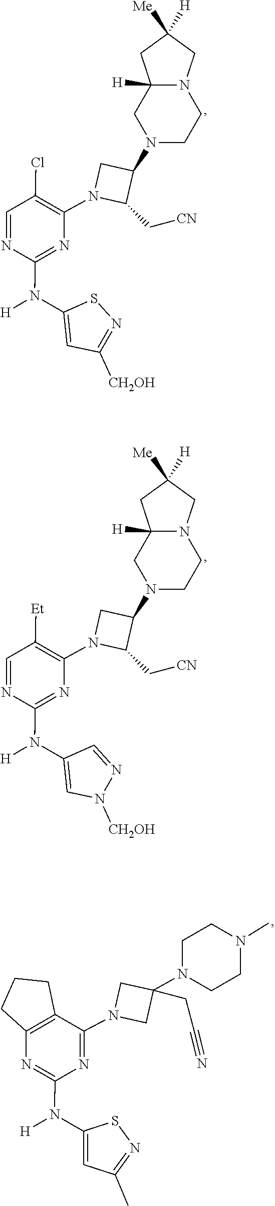 [SMILES: C[C@@H]1C[C@H]2CN([C@@H]3CN(c4nc(Nc5cc(CO)ns5)ncc4Cl)[C@H]3CC#N)CCN2C1 ; CCc1cnc(Nc2cnn(CO)c2)nc1N1C[C@@H](N2CCN3C[C@H](C)C[C@H]3C2)[C@@H]1CC#N ; Cc1cc(Nc2nc3c(c(N4CC(CC#N)(N5CCN(C)CC5)C4)n2)CCC3)sn1] 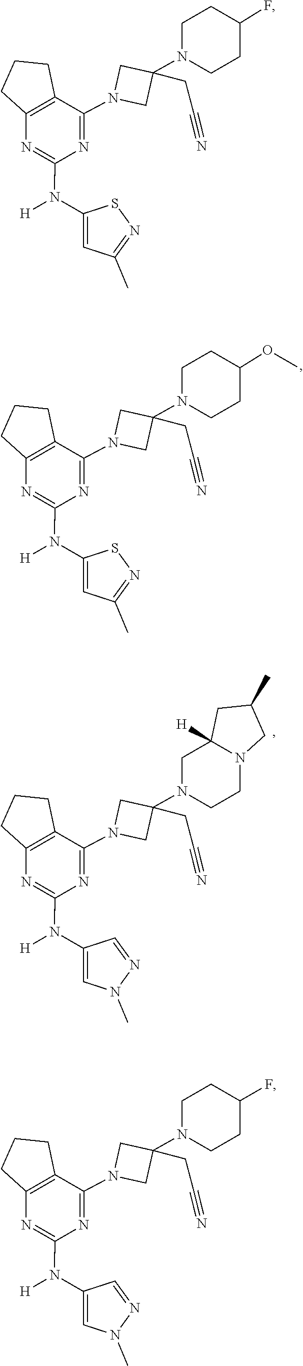 [SMILES: Cc1cc(Nc2nc3c(c(N4CC(CC#N)(N5CCC(F)CC5)C4)n2)CCC3)sn1 ; COC1CCN(C2(CC#N)CN(c3nc(Nc4cc(C)ns4)nc4c3CCC4)C2)CC1 ; C[C@@H]1C[C@H]2CN(C3(CC#N)CN(c4nc(Nc5cnn(C)c5)nc5c4CCC5)C3)CCN2C1 ; Cn1cc(Nc2nc3c(c(N4CC(CC#N)(N5CCC(F)CC5)C4)n2)CCC3)cn1] 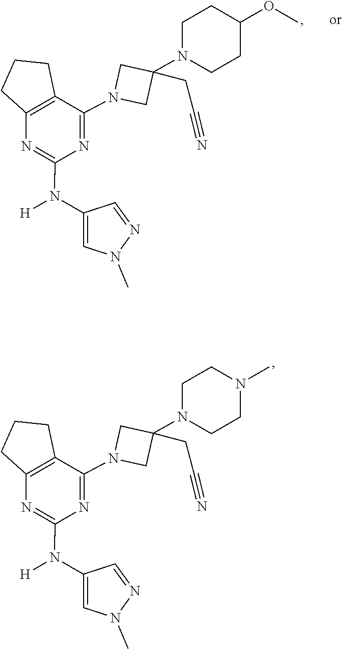 [SMILES: COC1CCN(C2(CC#N)CN(c3nc(Nc4cnn(C)c4)nc4c3CCC4)C2)CC1 ; CN1CCN(C2(CC#N)CN(c3nc(Nc4cnn(C)c4)nc4c3CCC4)C2)CC1]
or a pharmaceutically acceptable salt thereof.

11. The compound of claim 1, having a formula: 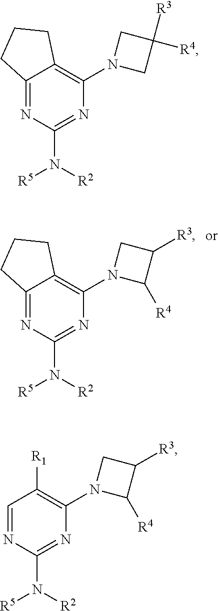
or a pharmaceutically acceptable salt thereof;
wherein
R⁵ is H, S(O)₂CH₃, or S(O)₂CH₂CH₃;
R¹ is H, CH₃, CH₂CH₃, cyclopropyl, OCH₃, F, Cl, or CN;
R² is 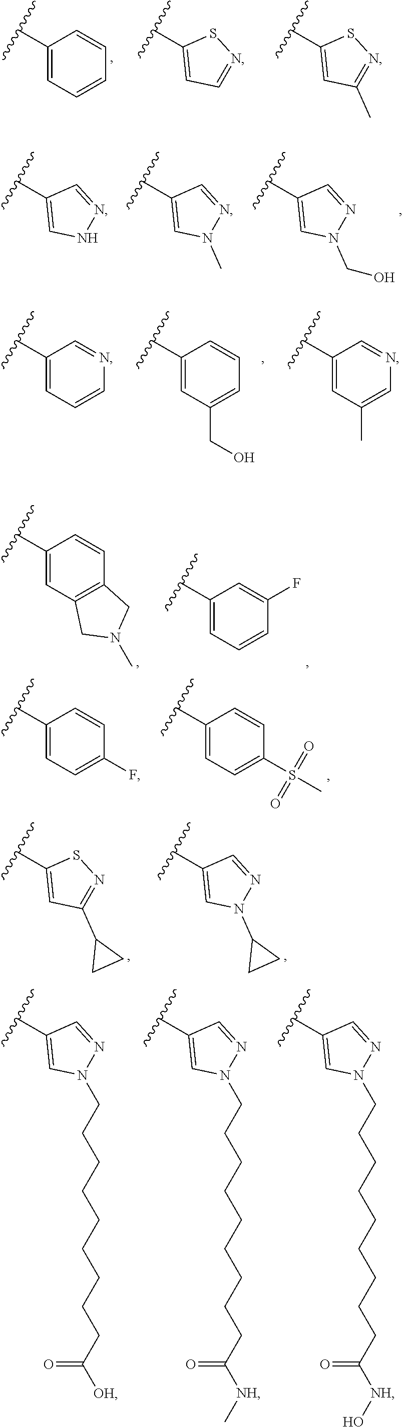 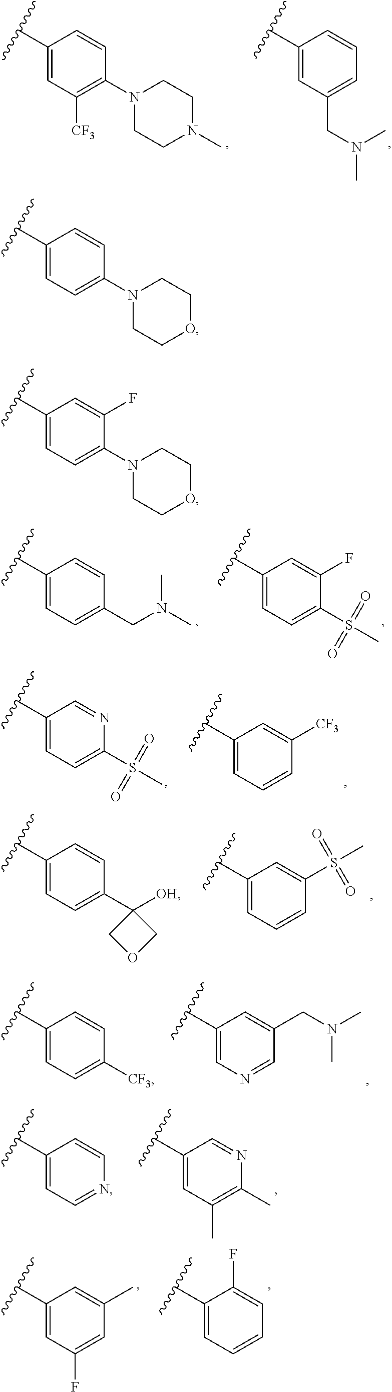 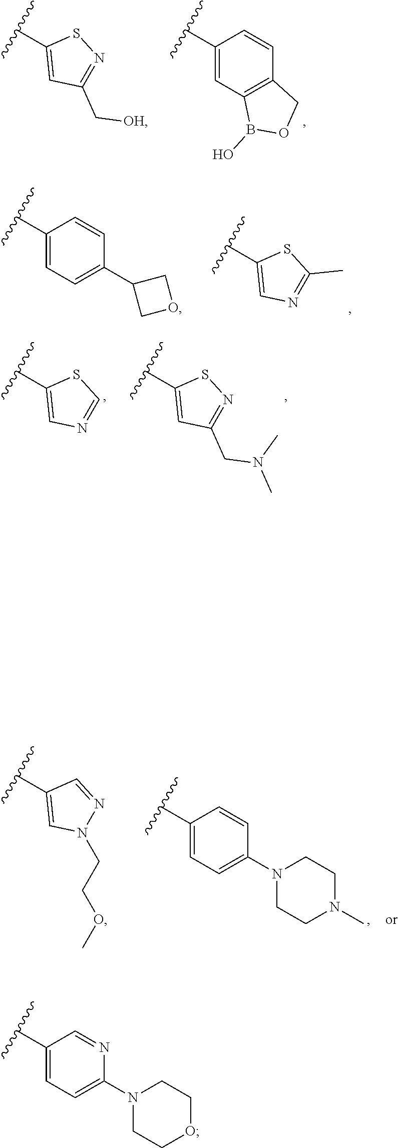
R³ is CN, OH, NH₂, 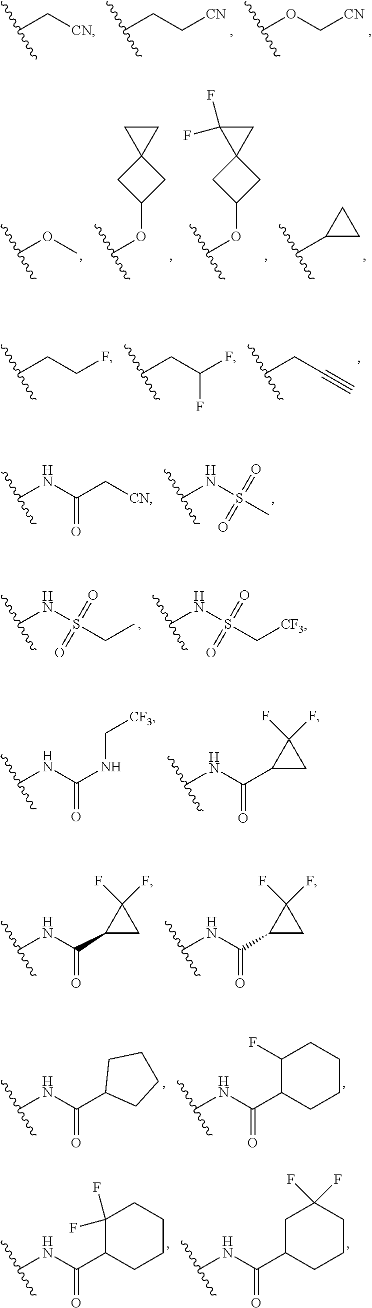 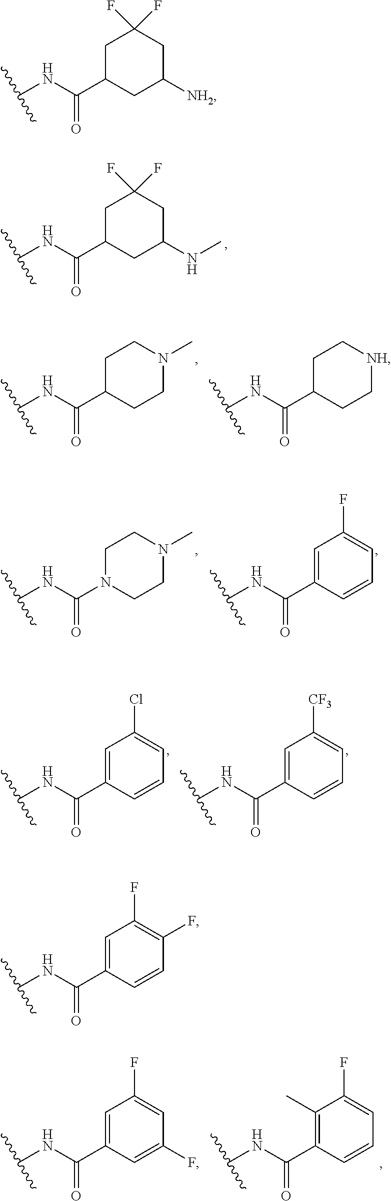 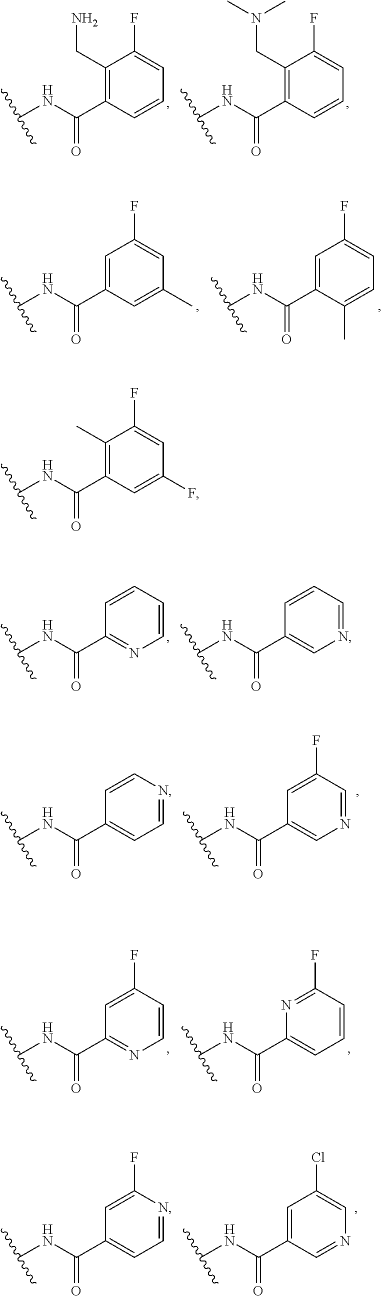 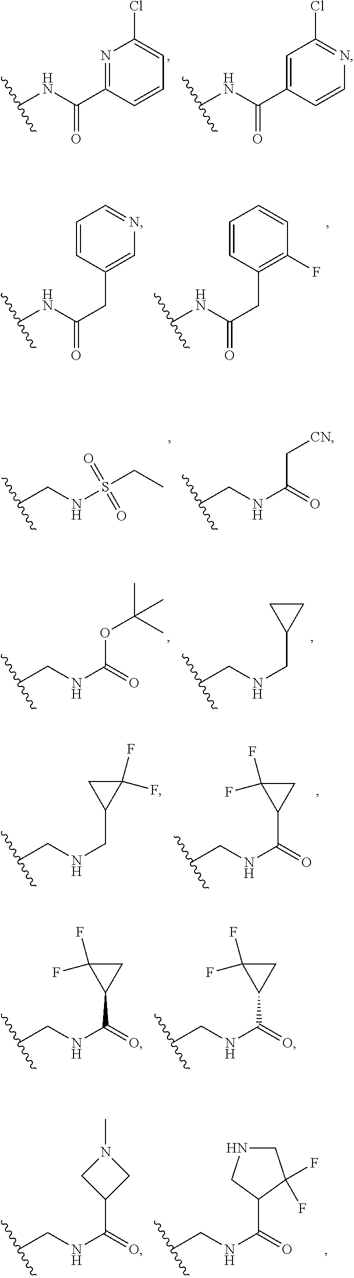 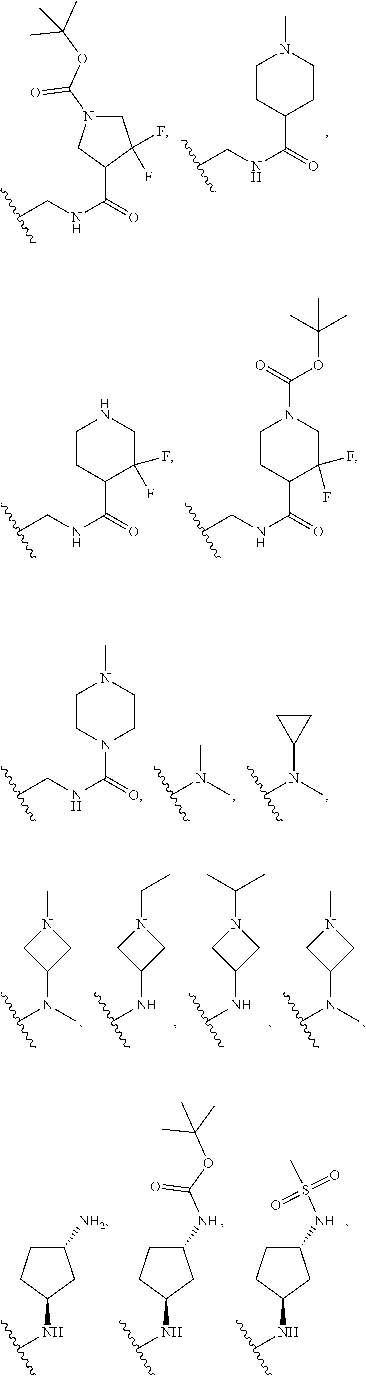 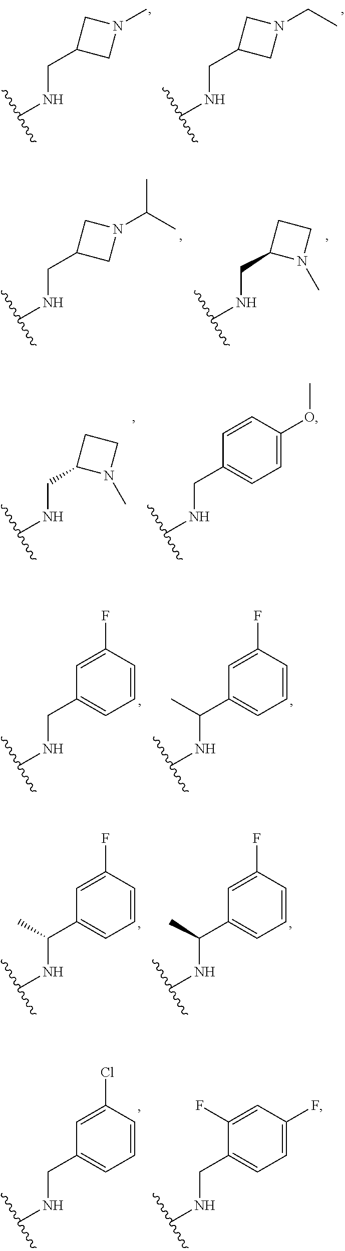 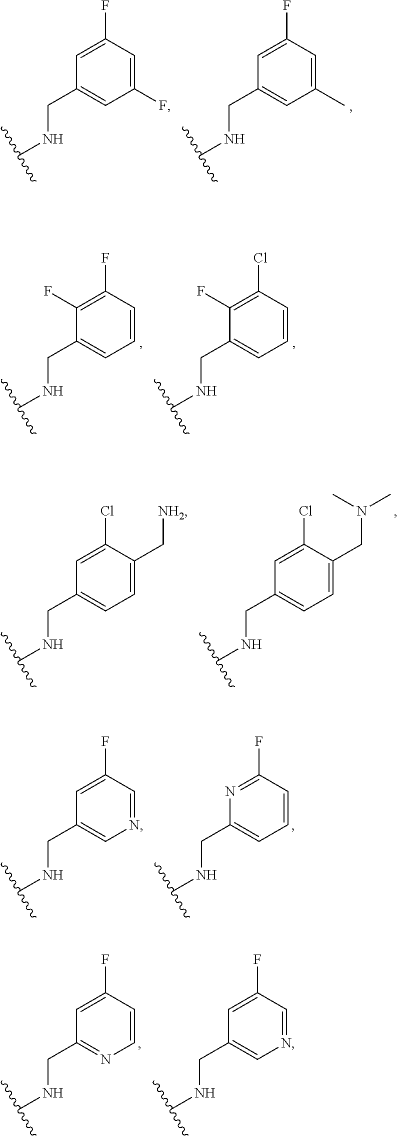 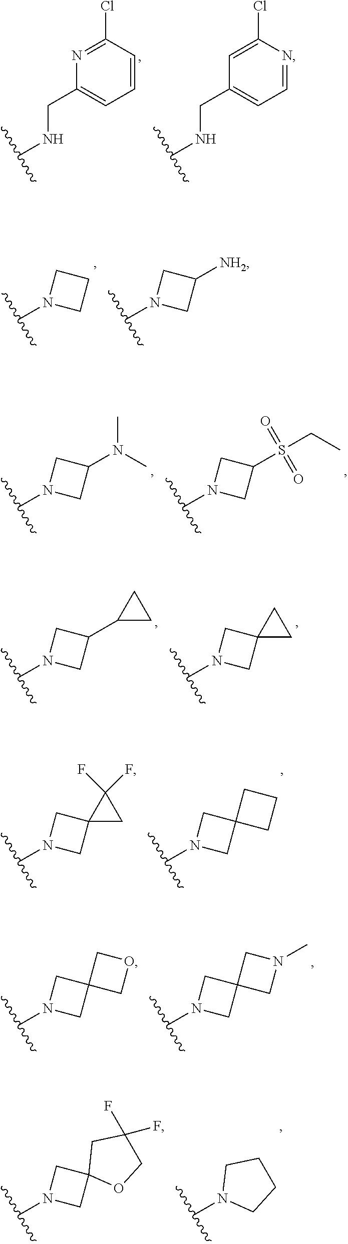 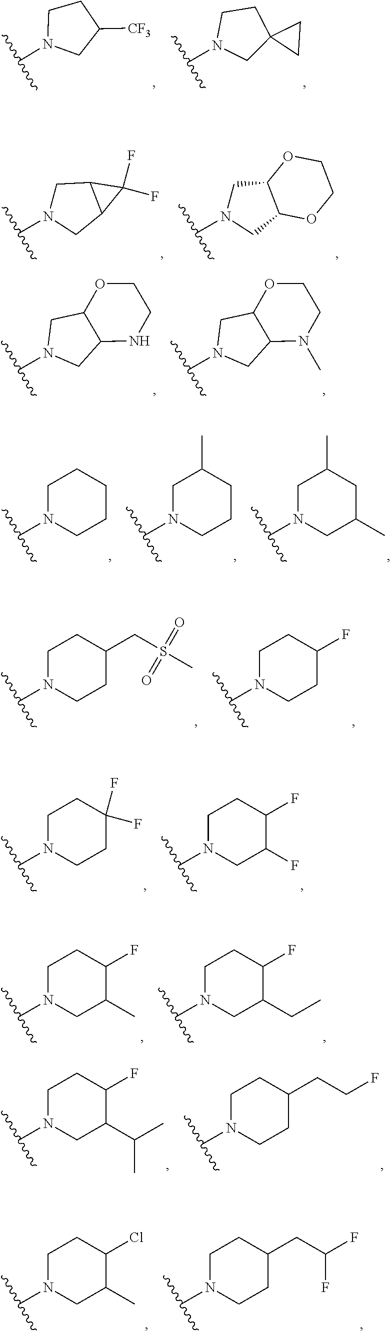 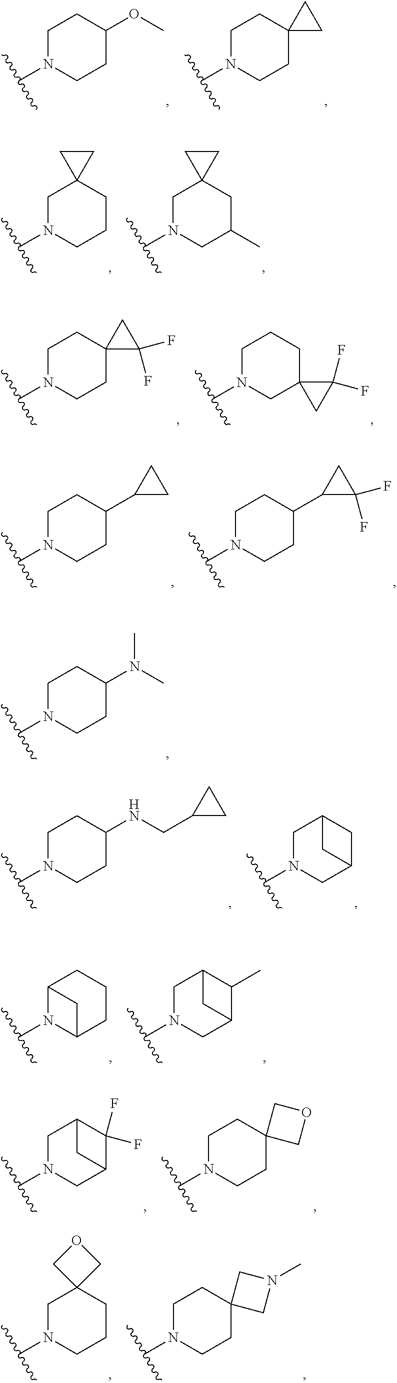 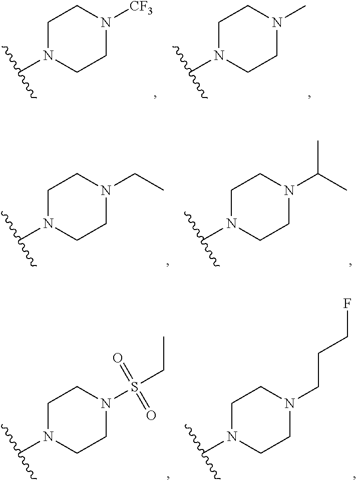 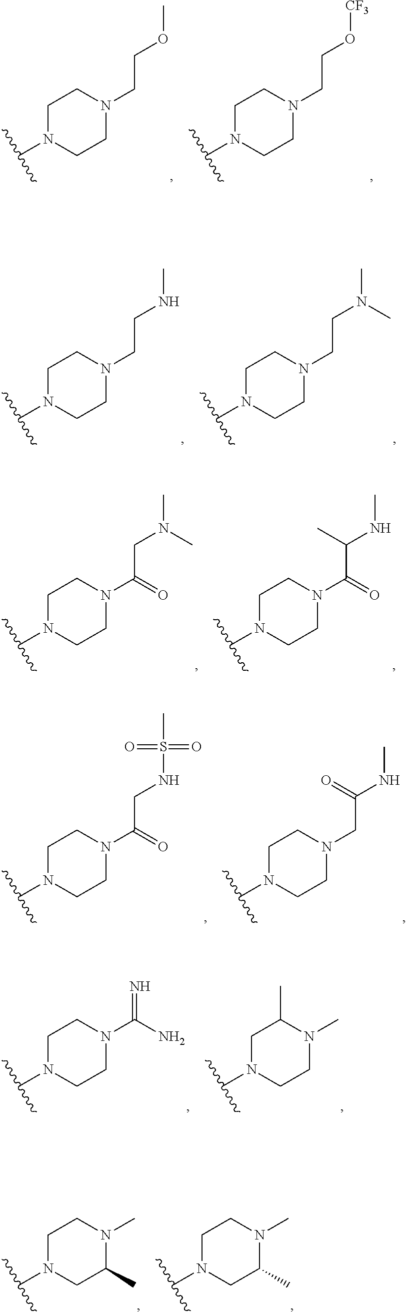 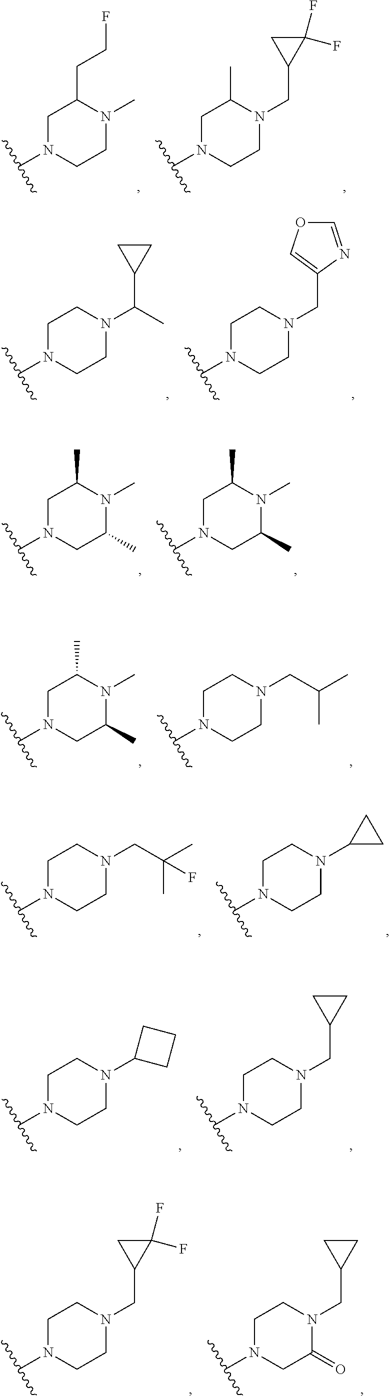 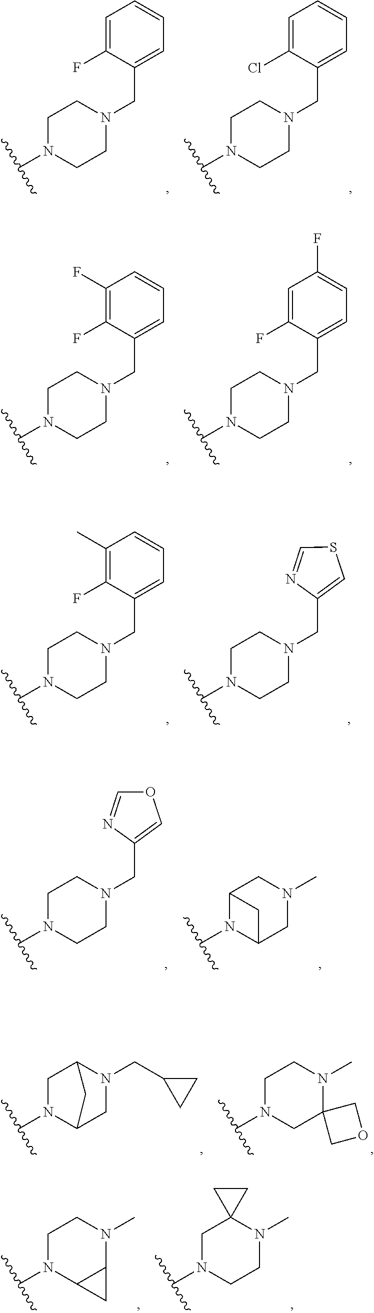 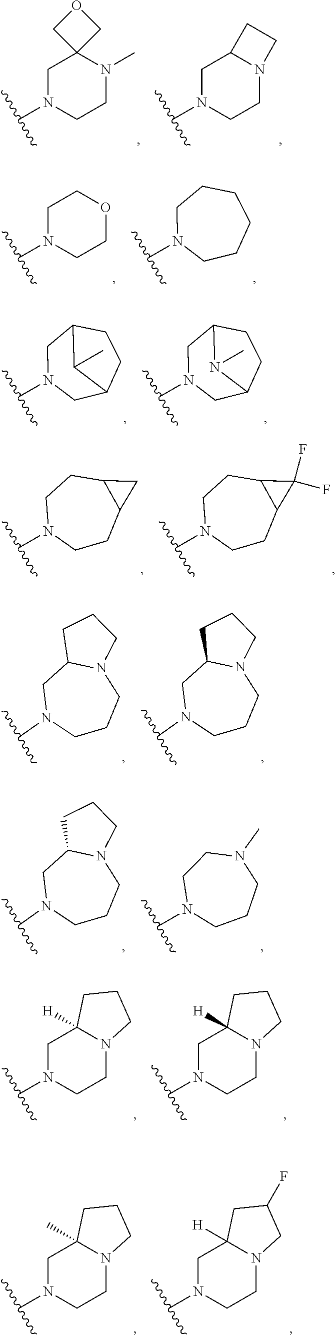 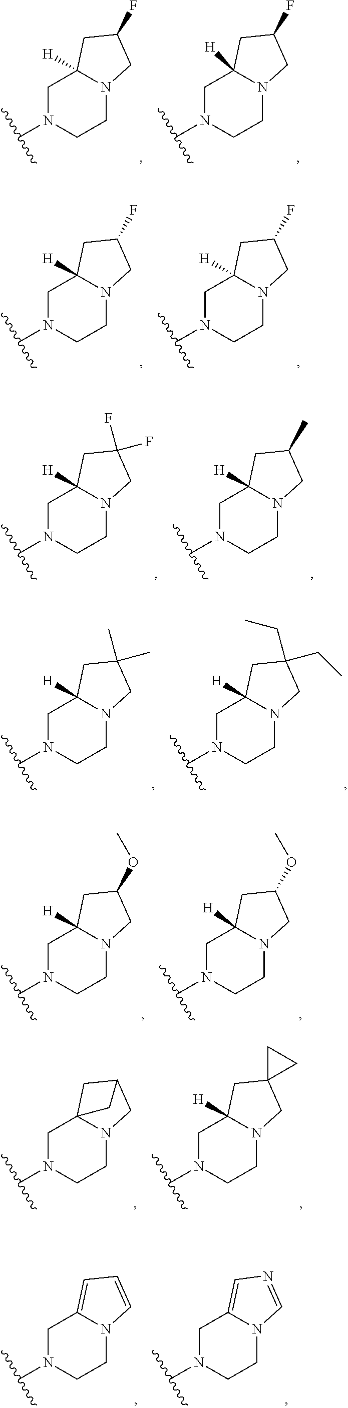 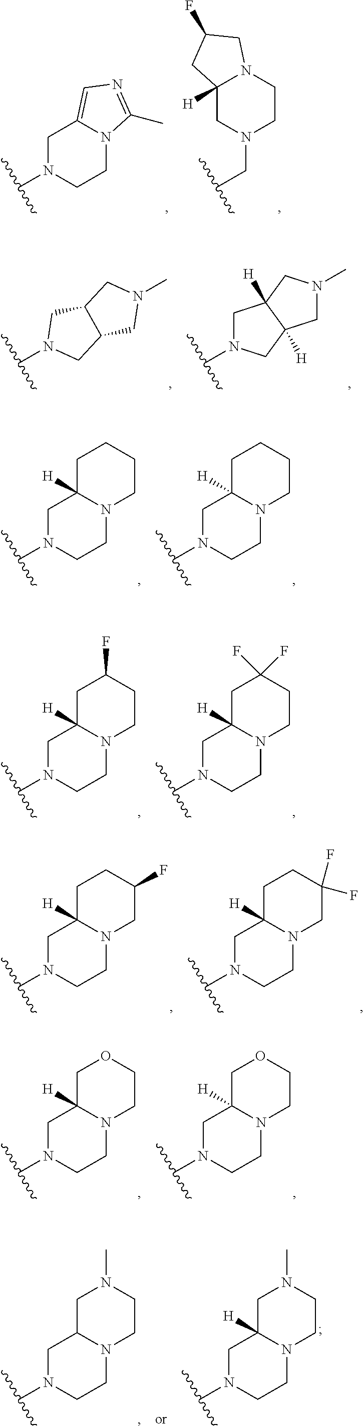
and
R⁴ is H, CH₃, CH₂CH₃, isopropyl, cyclopropyl, cyclobutyl, cyclopentyl, cyclohexyl, cycloheptyl, OH, CH₂OH, CH₂CH₂OH, CH₂F, CH₂CHF₂, CH₂CH₂F, CH₂CH₂Cl, CH₂-cyclopropyl, CH₂CN, CH₂CH₂CN, 3-fluorocyclobut-1-yl, tetrahydro-2H-pyran-4-yl, CH₂C(O)OCH₃, or CH₂OC(O)CH₃;
or R³ and R⁴, together with the atoms to which they are attached, combine to form

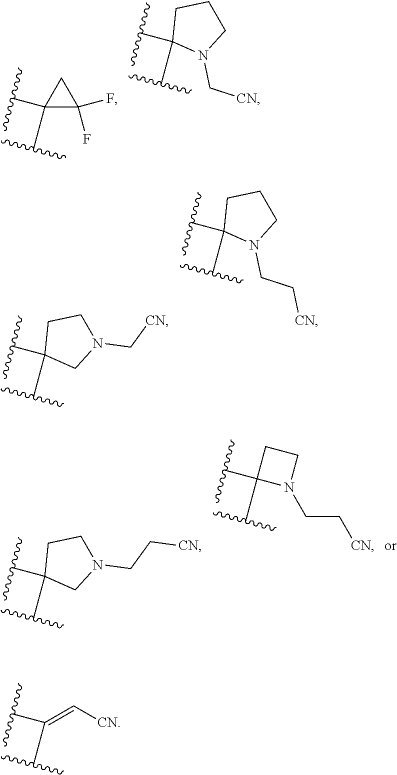

12. A composition, comprising the compound of claim 1.

13. The composition of claim 12, which is a pharmaceutical composition further comprising a pharmaceutically acceptable carrier.

14. A method treating a Janus kinase (JAK) related disease in a subject in need thereof, comprising administering to the subject a therapeutically effective amount of the compound of claim 1.

15. A method treating an eye disease in a subject in need thereof, comprising administering to the subject a therapeutically effective amount of the compound of claim 1.

16. A method of inhibiting Janus kinase (JAK) activity in a subject in need thereof, comprising administering to the subject a therapeutically effective amount of the compound of claim 1.

17. A method of inhibiting Janus kinase (JAK) activity in vitro, comprising contacting the JAK with an effective amount of the compound of claim 1.

18. A method, comprising administering the compound of claim 1 to a subject.

19. A kit, comprising the compound of claim 1, and instructions for use thereof.

20. The compound of claim 1, in a container.
